(19) United States
(12) Patent Application Publication
(10) Pub. No.: US 2026/0041677 A1
(43) Pub. Date: Feb. 12, 2026

(54) COMPOSITIONS AND METHODS FOR TREATING ADENOID CYSTIC CARCINOMA

(71) Applicant: ELEVAR THERAPEUTICS, INC., Fort Lee, NJ (US)

(72) Inventor: Seong Hoon Jang, Gaithersburg, MD (US)

(21) Appl. No.: 19/101,452

(22) PCT Filed: Jun. 2, 2023
(86) PCT No.: PCT/US2023/024351

§ 371 Date: Feb. 5, 2025

Related U.S. Application Data
(60) Provisional application No. 63/395,601, filed on Aug. 5, 2022.
(60) Provisional application No. 63/407,021, filed on Sep. 15, 2022.

(51) Int. Cl. A61K 31/444; A61P 35/00; A61P 35/04

(52) U.S. Cl. CPC A61K 31/444; A61P 35/00; A61P 35/04

(57) ABSTRACT

The disclosure is based, at least in part, on the discovery that if you treat recurrent or metastatic adenoid cystic carcinoma “RIM ACC”, in a subject who has one or more measurable ACC tumors/lesions that have increased in number and/or have increased in a dimension and/or volume by 20% or more prior to treatment, and/or who has an appearance of one or more new ACC tumors/lesions prior to treatment (also referred to herein as “severe RIM ACC”), by administering a high starting daily dosage (i.e., greater than 500 mg and up to about 1 000 mg), e.g., a starting dosage of about 700 mg, of rivoceranib or a pharmaceutically acceptable salt thereof, the severe RIM ACC in the subject improves with acceptable adverse events.

### CROSS-REFERENCE

This application claims the benefit of U.S. Provisional Application No. 63/395,601, filed on Aug. 5, 2022, and U.S. Provisional Application No. 63/407,021, filed on Sep. 15, 2022, the contents of which are incorporated herein by reference in their entireties.

### FIELD

The present disclosure relates to compositions and methods for treating adenoid cystic carcinoma (ACC), and in particular recurrent or metastatic ACC, in subjects.

### BACKGROUND

Tumor angiogenesis plays a critical role in malignant tumor growth and metastasis. When tumors grow beyond 1 mm³ angiogenesis, i.e., generation of vascular arborizations by budding from existing vessels, is necessary to provide enough blood for the survival of tumor cells. The growth speed and tendency of metastasis of tumors are associated with the level of neovascularization factors and the quantity of nascent microvessels. Since the hypothesis of “anti-angiogenesis therapy” was put forward by Dr. Folkman in the early 1970s, people have made considerable progress in this field, and inhibiting angiogenesis of tumors has been universally accepted as a promising anticancer strategy.

Adenoid cystic carcinoma (ACC) is a rare type of adenocarcinoma that arises from secretory glands, most commonly salivary glands. Tumor angiogenesis is believed to play a major role in progression of ACC. Expression of the receptor ligand VEGF by tumor cells correlates with tumor size and stage, vascular invasion, and increased risk of metastasis. It accounts for 25% of malignant tumors of the major salivary glands and 50% in minor salivary glands (including tongue, paranasal sinuses, palate, nasopharynx, lacrimal glands, and external auditory canal). ACC can also arise in secretory glands in other sites, such as the trachea, lung, and breast. Each year, about 1,300 people are diagnosed with ACC in the United States (Adenoid Cystic Carcinoma: Statistics. Cancer.net/adenoid-cystic-carcinoma/statistics. Published January 2021).

Most ACC primary tumors are treated with surgical resection. Despite good surgical technique, the 5-10-year recurrence rate is 30-75% (Boyle T. A., Laurent S., Semus S., Jeseph, N. Epidemiology of adenoid cystic carcinoma in the United States. Journal of Clinical Oncology. 2020; 38(15)). Postoperative radiotherapy is often given to try to reduce the recurrence rate. Nevertheless, recurrent and metastatic ACC (R/M ACC) are difficult to treat.

Rivoceranib (also known as YN968Dl, developed in China as apatinib and marketed as Aitan®) is an orally administered small molecule tyrosine kinase inhibitor with selectivity towards the VEGFR-2/kinase insert domain receptor. In tumor tissues, VEGF binds to VEGFR, stimulates VEGFR-mediated downstream signaling transduction, and ultimately leads to tumor angiogenesis. The classical VEGFRs in mammals include VEGFR1 Fms related receptor tyrosine kinase 1 (FLT1), VEGFR2 kinase insert domain receptor (KDR) and VEGFR3 (FLT4). Compared with the other 2 VEGFRs, VEGFR2 has higher affinity and kinase activity and is more important for direct regulation of angiogenesis, mitogenic signaling, and permeability-enhancing effects. Most VEGFRs are expressed by many tumor types, and their expression levels correlate with poor clinical outcomes. Rivoceranib selectively binds to and inhibits VEGFR2, which is believed to be principally responsible for inhibition of VEGF-stimulated endothelial cell migration and proliferation and decreases in tumor microvascular density. Rivoceranib has received approval in China, for treatment of advanced gastric cancer, and has received orphan medicinal product designation for the treatment of gastric cancer from Europe, the FDA, and the MFDS in South Korea.

### SUMMARY OF THE DISCLOSURE

The disclosure is based, at least in part, on the discovery that if you treat recurrent or metastatic adenoid cystic carcinoma (“R/M ACC”), in a subject who has one or more measurable ACC tumors/lesions that have increased in number and/or have increased in a dimension and/or volume by 20% or more prior to treatment, and/or who has an appearance of one or more new ACC tumors/lesions prior to treatment (also referred to herein as “severe R/M ACC”), by administering a high starting daily dosage (i.e., greater than 500 mg and up to about 1000 mg), e.g., a starting dosage of about 700 mg, of rivoceranib or a pharmaceutically acceptable salt thereof, the severe R/M ACC in the subject improves with acceptable adverse events. The present disclosure demonstrates that subjects receiving the relatively high starting dose of rivoceranib exhibited a significantly higher overall survival rate and a significant improvement of their disease progression and/or a significant slowing of disease growth even for severe R/M ACC, while tolerating adverse events. In cases in which the adverse events become intolerable, the starting dosage can be adjusted accordingly as described herein.

In a first aspect, the present disclosure provides methods of treating a subject having R/M ACC, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over 1, 2, 3, 4, 5, or 6 months prior to treatment. The methods include or consist of administering to the subject an initial or starting total daily amount of greater than 500 mg and up to about 1000 mg rivoceranib or a pharmaceutically acceptable salt thereof. In some embodiments, the subject had received VEGFR inhibitor therapy prior to treatment.

Also provided herein are methods of treating a subject having R/M ACC, the method including or consisting of assessing a level of severity of the R/M ACC of the subject; and administering to the subject a starting dose of a total daily amount of greater than 500 mg and up to about 1000 mg rivoceranib or a pharmaceutically acceptable salt thereof, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over 1, 2, 3, 4, 5, or 6 months prior to treatment.

Also provided herein are uses of compositions, and compositions for use, including a starting dose of a total daily amount of greater than 500 mg and up to about 1000 mg rivoceranib or a pharmaceutically acceptable salt thereof, for treating R/M ACC in a subject, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over 1, 2, 3, 4, 5, or 6 months prior to treatment.

Also provided herein are dosing regimens of rivoceranib or a pharmaceutically acceptable salt thereof for treatment of R/M ACC in a subject, comprising or consisting of administering to the subject a starting dosage of a total daily amount of greater than 500 mg and up to about 1000 mg rivoceranib or a pharmaceutically acceptable salt thereof for at least one cycle, wherein each cycle is at least 10 days, e.g., 28 days, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over 1, 2, 3, 4, 5, or 6 months prior to treatment.

Also provided herein are methods of treating R/M ACC in a subject; the methods include or consist of orally administering a starting dosage of a total daily amount of about 700 mg of rivoceranib or a mesylate salt thereof to the subject, wherein the starting dosage comprises one or more 100 mg tablets and one or more 200 mg tablets for the total daily amount of about 700 mg of rivoceranib or a mesylate salt thereof; wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over 1, 2, 3, 4, 5, or 6 months prior to treatment; and wherein the total daily amount of rivoceranib or a pharmaceutically acceptable salt thereof is down-titrated to a total daily amount of about 500 mg, about 300 mg, or about 200 mg of rivoceranib or a mesylate salt thereof, if the subject is experiencing serious adverse events.

Also provided herein are uses of a composition, and compositions for use, comprising a starting dosage of a total daily amount of greater than 500 mg to about 1000 mg rivoceranib or a pharmaceutically acceptable salt thereof, for treating adenoid cystic carcinoma in a subject, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over 1, 2, 3, 4, 5, or 6 months prior to treatment.

As used herein, the term “subject” is a vertebrate, such as a mammal, a fish, a bird, a reptile, or an amphibian. Thus, the subject of the methods disclosed herein can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are covered. In one aspect, the subject is a mammal. In one aspect, the subject is a human. In one aspect, the subject is a patient. A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation, such as adenoid cystic carcinoma.

As used herein, the term “treat,” “treating,” or “treatment” refers to the medical management of a subject with the intent to improve, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) inhibiting the disease, i.e., arresting its development; or (ii) relieving the disease, i.e., causing regression of the disease.

As used herein, the term “diagnosed or diagnosing” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be diagnosed or treated by a compound or composition that can inhibit uncontrolled cellular proliferation. Such a diagnosis can be in reference to a disorder, such as a disorder of uncontrolled cellular proliferation, cancer and the like, as discussed herein.

As used herein, the term “assessing” or “assessed” refers to a form of measurement, including determining if a disease or disorder is present or not, as well as, in some instances, determining the amount of something present and/or determining the change of the amount of something present over time. In some embodiments, assessing a subject can lead to a subject being identified to be in need of treatment for a disorder. As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the phrase “recurrent or metastatic adenoid cystic carcinoma” or “R/M ACC” refers to ACC that has either recurred in the part of the body of a subject where it originated or when the ACC has spread to cause tumors/lesions in a different part of the body other than where it originated and/or has tumors/lesions that have increased in size. As defined herein, “severe R/M ACC” means that the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over time prior to treatment as described herein, e.g., over 1, 2, 3, 4, 5, or 6 months prior to treatment. The subject may have been treated with other drugs than rivoceranib prior to treatment as described herein.

As used herein, the terms “administering” and “administration” refer to any type of method of providing a pharmaceutical preparation to a subject. Such types of methods are generally known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, intraurethral administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

As used herein, the term “pharmaceutically acceptable carrier” relates to nontoxic carriers or diluents, which are biologically compatible with subjects and are compositions commonly used to formulate pharmaceutical compositions for animal or human administration. Such carriers may include, however are not limited to, buffering agents, solubilizing agents, stabilizing agents or taste additives.

As used herein, the term “serious adverse events” refers to an adverse event that results in death, is life threatening, requires or prolongs hospitalization, causes persistent or significant disability or incapacity, or other conditions that represent significant hazards to the subject (Adverse Events are coded with Medical Dictionary for Regulatory Activities (MedDRA) Version 23.1). For example, serious adverse events can include respiratory, thoracic, mediastinal disorders, gastrointestinal disorders, infections and infestations, pneumonia, nervous system disorders, musculoskeletal and connective tissue disorders, or the like.

As used herein, “AUC” refers to the area under the curve, or the integral, of the plasma concentration of an active pharmaceutical ingredient or metabolite over time following a dosing event.

As used herein “AUC₀₋ₜ” is the integral under the plasma concentration curve from time 0 (dosing) to time “t”.

As used herein, “AUC_{0-∞}” is the AUC from time 0 (dosing) to time infinity. Unless otherwise stated, AUC refers to AUC_{0-∞}. Often a drug is packaged in a salt form, for example rivoceranib mesylate salt, and the dosage form strength refers to the mass of this salt form or the equivalent mass of the corresponding free base, rivoceranib.

As used herein, Cₘₐₓ is a pharmacokinetic parameter denoting the maximum observed blood plasma concentration following delivery of an active pharmaceutical ingredient. Cₘₐₓ occurs at the time of maximum plasma concentration, tₘₐₓ.

As used herein, “tₘₐₓ” is a pharmacokinetic parameter denoting the time to maximum blood plasma concentration following delivery of an active pharmaceutical ingredient

As used herein, “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

There are no approved systemic therapies for treatment of R/M ACC, and thus patients with R/M ACC, especially patients with severe R/M ACC as defined herein, are in urgent need of a therapy that demonstrates clinical benefit, prolonging time to disease progression or slowing disease growth, and ultimately improving survival, while being well-tolerated with minimal negative impact on quality of life. Because of the rarity of ACC, there are few clinical trials that have rigorously assessed the benefits of systemic therapy. Advantageously provided herein are methods of treating R/M ACC in subjects who have no approved therapeutic options, e.g., subjects who have severe R/M ACC, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over 1, 2, 3, 4, 5, or 6 months prior to treatment. Also, the present disclosure demonstrates that the methods disclosed herein have been shown to have reversed or slowed progression of the lesions in the subjects, measured by changes in tumor lesion diameters, even in subjects with severe R/M ACC. In addition, advantageously provided herein, the methods have been shown to increase the rate of overall survival of these subjects, and to have a tolerable safety profile and tolerable adverse events.

In this application, the use of the singular includes the plural unless specifically stated otherwise. It must be noted that, as used in the specification, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” are not limiting.

As used herein, ranges and amounts can be expressed as “about” a particular value or range. About also includes the exact amount. Hence “about 5 μl” means “about 5 μl” and also “5 μl.” Generally, the term “about” includes an amount that would be expected to be within experimental error. Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

Unless defined otherwise, all technical and scientific terms used herein have the same meaning as is commonly understood by one of skill in the art to which the claimed subject matter belongs. It is to be understood that the detailed description provides examples and explanations only, which are not restrictive of any subject matter claimed. All publications, patent applications, patents, and other references mentioned herein are incorporated by reference in their entirety. In case of conflict, the present specification, including definitions, will control. In addition, the materials, methods, and examples are illustrative only and not intended to be limiting.

Although methods and materials similar or equivalent to those described herein can be used in the practice or testing of the present invention, suitable methods and materials are described below. Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment.

Reference in the specification to “some embodiments,” “an embodiment,” “one embodiment,” or “other embodiments” means that a particular feature, structure, or characteristic described in connection with the embodiments is included in at least some embodiments, but not necessarily all embodiments, of the disclosures.

The section headings used herein are for organizational purposes only and are not to be construed as limiting the subject matter described.

Other features and advantages of the methods and compositions of the disclosure will be apparent from the following detailed description, and from the claims.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1 is a graph of patients with confirmed response per Response Evaluation Criteria in Solid Tumors Version 1.1 (RECIST V1.1) (efficacy evaluable population), wherein patients have severe R/M ACC (as defined herein) and are administered rivoceranib or a pharmaceutically acceptable salt thereof.

FIG. 2 is a graph of the maximum percent change in sum of target lesion diameters by RECIST V1.1 (efficacy evaluable population) evaluated by an investigator, wherein patients with the lesions have severe R/M ACC and are administered rivoceranib or a pharmaceutically acceptable salt thereof.

FIG. 3 is a graph of the maximum percent change in sum of target lesion diameters by RECIST V1.1 (efficacy evaluable population) evaluated by a blinded independent review committee (BIRC), wherein patients with the lesions have severe R/M ACC and are administered rivoceranib or a pharmaceutically acceptable salt thereof.

FIG. 4 is a graph of the percent change in sum of target lesion diameters per RECIST V1.1 (efficacy evaluable population) evaluated by an investigator, wherein patients with the lesions have severe R/M ACC and are administered rivoceranib or a pharmaceutically acceptable salt thereof.

FIG. 5 is a graph of the percent change in sum of target lesion diameters per RECIST V1.1 (efficacy evaluable population) evaluated by a BIRC, wherein patients with the lesions have severe R/M ACC and are administered rivoceranib or a pharmaceutically acceptable salt thereof.

FIG. 6 is a graph of the maximum percent change in sum of target lesion diameters for 6-month period prior to administration of rivoceranib and the first 6-months during administration of rivoceranib per RECIST V1.1 evaluated by an investigator for patients with greater than or equal to 20% increase in measurable lesions.

FIG. 7 is a graph of the percent change in sum of target lesion diameters for 6-month period prior to administration of rivoceranib and the first 6-months during administration of rivoceranib per RECIST V1.1 evaluated by an investigator (efficacy evaluable population).

FIG. 8 is a graph of patients having severe R/M ACC with a confirmed response to the administration of rivoceranib evaluated by a BIRC per CHOI response criteria (based on the journal article, Choi et al., Correlation of computed tomography and positron emission tomography in patients with metastatic gastrointestinal stromal tumor treated at a single institution with imatinib mesylate: proposal of new computed tomography response criteria. J Clin Oncol. 2007; 25(13): 1753-1759).

FIG. 9 is a graph of the duration of treatment of rivoceranib in patients as disclosed herein.

FIGS. 10A-10D are a series of computed tomography (CT) scan images showing reduction in tumor lesion density without reduction in tumor lesion size 2 months after starting treatment with rivoceranib in a patient as disclosed herein.

FIG. 11 is a graph of Kaplan-Meier Estimates of Progression-Free Survival per RECIST v1.1 Assessed by Investigator in the Efficacy Evaluable Population.

FIG. 12 is a graph of the treatment-emergent adverse events in patients with severe R/M ACC treated with rivoceranib as disclosed herein.

### DETAILED DESCRIPTION

Described herein are compositions and methods for treating severe R/M ACC, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over time prior to treatment as described herein, e.g., over 1, 2, 3, 4, 5, or 6 months prior to treatment. The subject may have been treated with other drugs than rivoceranib prior to treatment as described herein, or treated with a lower dose of rivoceranib, that was ineffective.

The methods include administering to the subject rivoceranib or a pharmaceutically acceptable salt thereof. In some embodiments, the methods include administering to the subject a starting dosage of a total daily amount of greater than 500 mg, e.g., 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 750 mg, 800 mg, 850 mg, 900 mg, 950 mg, and up to about 1000 mg rivoceranib (as a free base form) or a pharmaceutically acceptable salt thereof. In some embodiments, the methods include assessing a level of severity of the R/M ACC of the subject, and administering rivoceranib or a pharmaceutically acceptable salt thereof to the subject.

In some embodiments, the methods disclosed herein can improve adenoid cystic carcinoma in a subject, e.g., wherein the subject has measurable tumors/lesions that are increasing in number, or have increased in a dimension or volume by 20% or more, or has an appearance of new lesions, prior to treatment as described herein. In some embodiments, the methods can reduce tumor size in a subject. In some embodiments, the methods can suppress tumor growth in a subject. In some embodiments, the methods can suppress tumor growth in a subject, wherein the methods include administering to the subject a starting dosage of a total daily amount of at least or greater than 500 mg and up to about 1000 mg rivoceranib or a pharmaceutically acceptable salt thereof, e.g., wherein the subject has measurable tumors/lesions that are increasing in number, or have increased in a dimension or volume by 20% or more, or have an appearance of new lesions. In some embodiments, the methods can suppress tumor growth with no or minimal serious adverse events, wherein the methods include administering rivoceranib or a pharmaceutically acceptable salt thereof to a subject with adenoid cystic carcinoma. In some embodiments, the methods can suppress tumor growth with no treatment emergent serious adverse events, wherein the methods include administering rivoceranib or a pharmaceutically acceptable salt thereof to a subject with adenoid cystic carcinoma. In some embodiments, the methods can suppress tumor growth with no serious adverse events, wherein the methods include administering a starting dosage of a total daily amount of at least or greater than 500 mg and up to about 1000 mg rivoceranib or a pharmaceutically acceptable salt thereof to a subject with adenoid cystic carcinoma. In some embodiments, the methods can suppress tumor growth with no treatment emergent serious adverse events, wherein the methods include administering a starting dosage of a total daily amount of about at least or greater than 500 mg and up to about 1000 mg rivoceranib or a pharmaceutically acceptable salt thereof to a subject with adenoid cystic carcinoma.

As described herein, the starting dosage may be modified over time, depending on the specific patient's or subject's tolerance to the higher dosage.

In some embodiments, the R/M ACC of the subject is recurrent ACC. In some embodiments, the R/M ACC of the subject is metastatic ACC.

In some embodiments, the R/M ACC of the subject comprises one or more lesions. In some embodiments, the lesion is measured before the treatment and either during the treatment or after the treatment is concluded, or both. In some embodiments, the lesion is measured using radiological assessments, e.g., using a computerized tomography scan or magnetic resonance imaging. In some embodiments, the lesion has reduced in size during and/or after the treatment. In some embodiments, the methods include assessing the ACC of the subject, wherein one or more lesions are measured before treatment. In some embodiments, the methods include assessing the adenoid cystic carcinoma in a subject, wherein assessing includes measuring one or more lesions in the subject, optionally measuring one or more lesions in the subject over a period of time, e.g., every week, every two weeks, every three weeks, once per month, once every two months, once every three months, once every four months, once every five months, once every six months, or more.

In some embodiments, the methods of improving and/or treating R/M ACC, e.g., severe R/M ACC, include assessing the R/M ACC of a subject, wherein the subject has one or more lesions that are increasing in size, e.g., an area or volume. In some embodiments, the total measurable lesions of the subject are increasing in size by at least 10% over a period of time, e.g., about 1 week, 1 month, 2 month, 3 months, 4 months, 5 months, 6 months, or more. In some embodiments, the total measurable lesions of the subject have increased in size by at least 20% over a period of time prior to treatment as described herein. In some embodiments, the total measurable lesions of the subject are increasing in size by at least 25% over a period of time. In some embodiments, the total measurable lesions of the subject are increasing in size by at least 30% over a period of time. In some embodiments, the total measurable lesions of the subject are increasing in size by at least 40% over a period of time. In some embodiments, the total measurable lesions of the subject are increasing in size by at least 50% over a period of time. In some embodiments, the total measurable lesions of the subject are increasing in size by at least 60% over a period of time. In some embodiments, the total measurable lesions of the subject are increasing in size by at least 70% over a period of time. In some embodiments, the total measurable lesions of the subject are increasing in size by at least 80% over a period of time. In some embodiments, the total measurable lesions of the subject are increasing in size by at least 90% or more over a period of time.

In some embodiments, the methods of treating R/M ACC in a subject result in reducing the size, e.g., an area or volume, of measurable lesions in a subject. In some embodiments, the lesion has reduced in size by at least 10%. In some embodiments, the lesion has reduced in size by at least 20%. In some embodiments, the lesion has reduced in size by at least 25%. In some embodiments, the lesion has reduced in size by at least 30%. In some embodiments, the lesion has reduced in size by at least 40%. In some embodiments, the lesion has reduced in size by at least 50%. In some embodiments, the lesion has reduced in size by at least 60%. In some embodiments, the lesion has reduced in size by at least 70%. In some embodiments, the lesion has reduced in size by at least 75%. In some embodiments, the lesion has reduced in size by at least 80%. In some embodiments, the lesion has reduced in size by at least 90%.

In some embodiments, the methods disclosed herein are a first line of therapy for treating ACC, e.g., severe R/M ACC. In some embodiments, the methods are a second line of therapy, a third line of therapy, or a fourth line of therapy after the prior treatment for the ACC has failed, substantially failed, or the disease is substantially refractory to the first line therapy. In some embodiments, a patient has received at least one line of therapy for treating ACC prior to being administered rivoceranib or a pharmaceutically acceptable salt thereof. In some embodiments, the prior line of therapy may be a line of chemotherapy or immunotherapy.

It is understood that ACC refers to or describes the physiological condition in mammals that is typically characterized by unregulated cell growth. The ACC may be multi-drug resistant (MDR) or drug-sensitive.

### Rivoceranib (Apatinib)

Generally, rivoceranib is an organic heterocyclic chemical compound of having the following chemical formula:

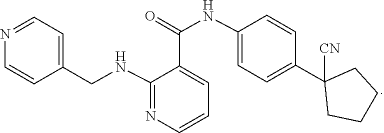 [SMILES: N#CC1(c2ccc(NC(=O)c3cccnc3NCc3ccncc3)cc2)CCCC1]

Rivoceranib (chemical name N-[4-(1-cyanocyclopentyl)phenyl]-2-{[(pyridin-4-yl)methyl]amino}pyridine-3-carboxamide, also known as YN968Dl, developed in China as apatinib and marketed as Aitan®) is an orally administered small molecule tyrosine kinase inhibitor. It selectively inhibits vascular endothelial growth factor receptor (VEGFR)-2 leading to blockage of tumor vascular angiogenesis, diminishes survival of existing blood vessels, and retards growth of tumors. Proliferation of endothelial cells is targeted directly, and inhibition of the release of proangiogenic growth factors by cancer or stromal cells is targeted indirectly.

In some embodiments, the methods for treating adenoid cystic carcinoma in a subject having adenoid cystic carcinoma include administering to the subject a total daily amount of rivoceranib or a pharmaceutically acceptable salts thereof such that the adenoid cystic carcinoma of the subject is improved, wherein the subject has measurable tumors/lesions that are increasing in number, or have increased in a dimension or volume by 20% or more, or have an appearance of new lesions prior to treating.

In some embodiments, the methods for treating ACC, e.g., R/M ACC, e.g., severe R/M ACC, in a subject having ACC include administering to the subject a total daily amount of greater than 500 mg and up to about 700 mg rivoceranib or a pharmaceutically acceptable salt thereof, wherein the subject has measurable tumors/lesions that are increasing in number, or have increased in a dimension or volume by 20% or more, or have an appearance of new lesions prior to treating. In some embodiments, the adenoid cystic carcinoma is improved. As disclosed herein, the amount (e.g., the mass) of rivoceranib or a pharmaceutically acceptable salt thereof is provided throughout this disclosure as the rivoceranib free base form, even though the rivoceranib can be administered as a pharmaceutically acceptable salt thereof. For example, when the disclosure describes the amount of rivoceranib or a pharmaceutically acceptable salt thereof administered is about 700 mg, the actual drug administered could be 700 mg of rivoceranib free base or the corresponding amount of rivoceranib pharmaceutically acceptable salt thereof, such as, about 869 mg of rivoceranib mesylate salt.

In some embodiments, the rivoceranib is administered in the form of a free base. In some embodiments, the rivoceranib is administered in the form of a pharmaceutically acceptable salt, such as a mesylate salt form, e.g., having the following chemical structure:

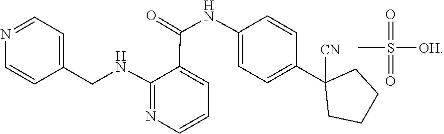

The rivoceranib mesylate has a CAS Registry no. 1218779-75-9, molecular weight of 493.58, and empirical formula C₂₅H₂₇N₅O₄S.

Suitable pharmaceutically acceptable salts will be apparent to those skilled in the art and include those described in J. Pharm. Sci., 1977, 66, 1-19, such as acid addition salts formed with inorganic acid e.g. hydrochloric, hydrobromic, sulphuric, nitric or phosphoric acid; and organic acids, e.g. succinic, maleic, acetic, fumaric, citric, tartaric, benzoic, p-toluenesulfonic, methanesulfonic or naphthalenesulfonic acid. In some embodiments, the pharmaceutically acceptable salt includes one or more of a mesylate salt, hydrochloride salt, sulfate salt, phosphate salt, maleate salt, citrate salt, or succinate salt. In some embodiments, the pharmaceutically acceptable salt is a mesylate salt.

In some embodiments, the rivoceranib or a pharmaceutically acceptable salt thereof is administered in a starting dosage of at least, i.e., great than 500 mg per day. In some embodiments, the rivoceranib or a pharmaceutically acceptable salt thereof is administered, e.g., in a total daily dose, in an amount of at least about 525 mg. In some embodiments, the rivoceranib or a pharmaceutically acceptable salt thereof is administered, e.g., in a total daily dose, in an amount of at least about 550 mg. In some embodiments, the rivoceranib or a pharmaceutically acceptable salt thereof is administered, e.g., in a total daily dose, in an amount of at least about 575 mg. In some embodiments, the rivoceranib or a pharmaceutically acceptable salt thereof is administered, e.g., in a total daily dose, in an amount of at least about 600 mg. In some embodiments, the rivoceranib or a pharmaceutically acceptable salt thereof is administered, e.g., in a total daily dose, in an amount of at least about 625 mg. In some embodiments, the rivoceranib or a pharmaceutically acceptable salt thereof is administered, e.g., in a total daily dose, in an amount of at least about 650 mg. In some embodiments, the rivoceranib or a pharmaceutically acceptable salt thereof is administered, e.g., in a total daily dose, in an amount of at least about 675 mg. In some embodiments, the rivoceranib or a pharmaceutically acceptable salt thereof is administered, e.g., in a total daily dose, in an amount of up to 1000 mg. In some embodiments, the rivoceranib or a pharmaceutically acceptable salt thereof is administered in an amount of at least 500 mg and up to 1000 mg. In some embodiments, the rivoceranib or a pharmaceutically acceptable salt thereof is administered in an amount of at least 500 mg and up to 900 mg. In some embodiments, the rivoceranib or a pharmaceutically acceptable salt thereof is administered in an amount of at least 500 mg and up to 800 mg. In some embodiments, the rivoceranib or a pharmaceutically acceptable salt thereof is administered in an amount of at least 500 mg and up to 700 mg. In some embodiments, the rivoceranib or a pharmaceutically acceptable salt thereof is administered in an amount of at least about 510 mg and up to 700 mg. In some embodiments, the rivoceranib or a pharmaceutically acceptable salt thereof is administered in an amount of at least about 525 mg and up to 700 mg. In some embodiments, the rivoceranib or a pharmaceutically acceptable salt thereof is administered in an amount of at least about 550 mg and up to 700 mg. In some embodiments, the rivoceranib or a pharmaceutically acceptable salt thereof is administered in an amount of at least about 575 mg and up to 700 mg. In some embodiments, the rivoceranib or a pharmaceutically acceptable salt thereof is administered in an amount of at least about 600 mg and up to 700 mg. In some embodiments, the rivoceranib or a pharmaceutically acceptable salt thereof is rivoceranib free base or rivoceranib mesylate salt.

In some embodiments, the methods of treating disclosed herein can include titration. As used herein, “titration” refers to a method to limit potential side effects in a subject by taking time to see how the subject reacts to a drug, e.g., rivoceranib. In this disclosure, titration is used after administration of the medication at a high starting dose, e.g., about 700 mg daily, and the subject's adverse events, if any, are assessed. If the subject is experiencing serious adverse events or intolerable adverse events, the dose is reduced (e.g., down-titrated) until either the serious adverse events are diminished or gone, or if the adverse events become tolerable. In some embodiments, the total daily amount of rivoceranib or a pharmaceutically acceptable salt thereof is titrated. In some embodiments, the total daily amount of rivoceranib or a pharmaceutically acceptable salt thereof is down-titrated after the subject was administered a total daily amount rivoceranib or a pharmaceutically acceptable salt thereof of at least about 500 mg and up to about 1000 mg, e.g., 1000 mg, at least once or for at least two, three, four, five, six, or seven days.

In some embodiments, the total daily amount of rivoceranib or a pharmaceutically acceptable salt thereof is reduced if the subject is experiencing serious adverse events. In some embodiments, the total daily amount of rivoceranib or a pharmaceutically acceptable salt thereof is down-titrated to about 100 mg. In some embodiments, the total daily amount of rivoceranib or a pharmaceutically acceptable salt thereof is down-titrated to about 150 mg. In some embodiments, the total daily amount of rivoceranib or a pharmaceutically acceptable salt thereof is down-titrated to about 200 mg. In some embodiments, the total daily amount of rivoceranib or a pharmaceutically acceptable salt thereof is down-titrated to about 225 mg. In some embodiments, the total daily amount of rivoceranib or a pharmaceutically acceptable salt thereof is down-titrated to about 250 mg. In some embodiments, the total daily amount of rivoceranib or a pharmaceutically acceptable salt thereof is down-titrated to about 275 mg.

In some embodiments, the total daily amount of rivoceranib or a pharmaceutically acceptable salt thereof is down-titrated to about 300 mg. In some embodiments, the total daily amount of rivoceranib or a pharmaceutically acceptable salt thereof is down-titrated to about 325 mg. In some embodiments, the total daily amount of rivoceranib or a pharmaceutically acceptable salt thereof is down-titrated to about 350 mg. In some embodiments, the total daily amount of rivoceranib or a pharmaceutically acceptable salt thereof is down-titrated to about 375 mg.

In some embodiments, the total daily amount of rivoceranib or a pharmaceutically acceptable salt thereof is down-titrated to about 400 mg. In some embodiments, the total daily amount of rivoceranib or a pharmaceutically acceptable salt thereof is down-titrated to about 425 mg. In some embodiments, the total daily amount of rivoceranib or a pharmaceutically acceptable salt thereof is down-titrated to about 450 mg. In some embodiments, the total daily amount of rivoceranib or a pharmaceutically acceptable salt thereof is down-titrated to about 475 mg.

In some embodiments, the total daily amount of rivoceranib or a pharmaceutically acceptable salt thereof is down-titrated to about 500 mg. In some embodiments, the total daily amount of rivoceranib or a pharmaceutically acceptable salt thereof is down-titrated to about 525 mg. In some embodiments, the total daily amount of rivoceranib or a pharmaceutically acceptable salt thereof is down-titrated to about 550 mg. In some embodiments, the total daily amount of rivoceranib or a pharmaceutically acceptable salt thereof is down-titrated to about 575 mg.

In some embodiments, the total daily amount of rivoceranib or a pharmaceutically acceptable salt thereof is down-titrated to about 600 mg. In some embodiments, the total daily amount of rivoceranib or a pharmaceutically acceptable salt thereof is down-titrated to about 625 mg. In some embodiments, the total daily amount of rivoceranib or a pharmaceutically acceptable salt thereof is down-titrated to about 650 mg. In some embodiments, the total daily amount of rivoceranib or a pharmaceutically acceptable salt thereof is down-titrated to about 675 mg.

### Pharmaceutical Compositions

In one aspect, the disclosure relates to pharmaceutical compositions comprising the compounds of the disclosure. That is, a pharmaceutical composition can be provided comprising a therapeutically effective amount of rivoceranib or a pharmaceutically acceptable salt thereof. In certain aspects, the disclosed pharmaceutical compositions comprise the disclosed compounds (such as rivoceranib or a pharmaceutically acceptable salt thereof) as active ingredients, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the compounds of the disclosure, or pharmaceutically acceptable salts thereof, of this disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion.

In some embodiments, the rivoceranib or a pharmaceutically acceptable salt thereof is administered in an oral liquid, solid, or semisolid dosage form. In some embodiments, the rivoceranib or a pharmaceutically acceptable salt thereof is administered as a solid oral dosage form. In some embodiments, the rivoceranib or a pharmaceutically acceptable salt thereof is administered as a pill, tablet, chewable tablet, specialty tablet, buccal tablet, sub-lingual tablet, orally-disintegrating tablet, capsule, gel capsule, soft gel capsule, hard gel capsule, sachet, powder, granule, crystal or orally dispersible film. In some embodiments, the rivoceranib or a pharmaceutically acceptable salt thereof is administered as a dried powder, a liquid, a capsule, a pellet or a tablet. In some embodiments, the rivoceranib or a pharmaceutically acceptable salt thereof is administered as a tablet. In some embodiments, the rivoceranib or a pharmaceutically acceptable salt thereof is administered as a film coated tablet. In addition to the common dosage forms set out above, the compounds of the disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical compositions of this disclosure can include a pharmaceutically acceptable carrier, and rivoceranib or a pharmaceutically acceptable salt thereof. The compounds of the disclosure, or pharmaceutically acceptable salts thereof, can also be included in pharmaceutical compositions in combination with one or more other therapeutically active compounds. In such embodiments, wherein the rivoceranib or a pharmaceutically acceptable salt thereof is administered as a solid oral dosage form, the rivoceranib or a pharmaceutically acceptable salt thereof may be admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alginates, gelatin, polyvinylpyrrolidone, sucrose, and gum acacia, (c) humectants, as for example, glycerol, (d) disintegrating agents, as for example, agar-, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, acetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

In some embodiments, solid dosage forms may be prepared with coatings and shells, such as enteric coatings and others known in the art. They may contain pacifying agents, and can also be of such composition that they release the rivoceranib or a pharmaceutically acceptable salt thereof in a certain part of the intestinal tract in a delayed manner. Examples of embedded compositions that can be used are polymeric substances and waxes. The rivoceranib or a pharmaceutically acceptable salt thereof may also be in microencapsulated form, if appropriate, with one or more of the above-mentioned excipients.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. In some embodiments, the rivoceranib or a pharmaceutically acceptable salt thereof is administered as a liquid oral dosage form. In some embodiments, the rivoceranib or a pharmaceutically acceptable salt thereof is administered as a solution, suspension, drink, syrup, elixir, ampoule, dispersion, semi-solid or soft gel. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this disclosure can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In some embodiments, the tablet comprises the rivoceranib or a pharmaceutically acceptable salt thereof in an amount of about 100 mg. In some embodiments, the tablet comprises the rivoceranib or a pharmaceutically acceptable salt thereof in an amount of about 150 mg. In some embodiments, the tablet comprises the rivoceranib or a pharmaceutically acceptable salt thereof in an amount of about 200 mg. In some embodiments, the tablet comprises the rivoceranib or a pharmaceutically acceptable salt thereof in an amount of about 250 mg. In some embodiments, the tablet comprises the rivoceranib or a pharmaceutically acceptable salt thereof in an amount of about 300 mg. In some embodiments, the tablet comprises the rivoceranib or a pharmaceutically acceptable salt thereof in an amount of about 350 mg. In some embodiments, the tablet comprises the rivoceranib or a pharmaceutically acceptable salt thereof in an amount of about 400 mg. In some embodiments, the tablet comprises the rivoceranib or a pharmaceutically acceptable salt thereof in an amount of about 450 mg. In some embodiments, the tablet comprises the rivoceranib or a pharmaceutically acceptable salt thereof in an amount of about 500 mg. In some embodiments, the tablet comprises the rivoceranib or a pharmaceutically acceptable salt thereof in an amount of about 550 mg. In some embodiments, the tablet comprises the rivoceranib or a pharmaceutically acceptable salt thereof in an amount of about 600 mg. In some embodiments, the tablet comprises the rivoceranib or a pharmaceutically acceptable salt thereof in an amount of about 650 mg. In some embodiments, the tablet further comprises one or more of pregelatinized starch, microcrystalline cellulose, sodium starch glycolate, povidone (K-30), colloidal silicon dioxide, magnesium stearate and Opadry white.

In some embodiments, the rivoceranib or a pharmaceutically acceptable salt thereof is administered in one or more 100 mg rivoceranib tablets. In some embodiments, the rivoceranib or a pharmaceutically acceptable salt thereof is administered in five, six or seven 100 mg rivoceranib tablets. In some embodiments, the rivoceranib or a pharmaceutically acceptable salt thereof is administered in one or more 200 mg rivoceranib tablets. In some embodiments, the rivoceranib or a pharmaceutically acceptable salt thereof is administered in three or four 200 mg rivoceranib tablets. In some embodiments, the rivoceranib or a pharmaceutically acceptable salt thereof is administered in two 200 mg rivoceranib tablets and two 100 mg rivoceranib tablets. In some embodiments, the rivoceranib or a pharmaceutically acceptable salt thereof is administered in one 200 mg rivoceranib tablet and five 100 mg rivoceranib tablets. In some embodiments, the rivoceranib or a pharmaceutically acceptable salt thereof is administered in two 200 mg rivoceranib tablets and three 100 mg rivoceranib tablets. In some embodiments, the rivoceranib or a pharmaceutically acceptable salt thereof is administered in three 200 mg rivoceranib tablets and one 100 mg rivoceranib tablets.

In some embodiments, each tablet has a core and a coating. In some embodiments, the core comprises one or more of colloidal silicone dioxide, microcrystalline cellulose, magnesium stearate, povidone, pregelatinized starch 1500, and sodium starch glycolate. In some embodiments, the core comprises colloidal silicone dioxide, microcrystalline cellulose, magnesium stearate, povidone, pregelatinized starch 1500, and sodium starch glycolate. In some embodiments, the coating comprises one or more of hypromellose, polyethylene glycol, polysorbate 80, and titanium dioxide. In some embodiments, the coating comprises hypromellose, polyethylene glycol, polysorbate 80, and titanium dioxide.

The pharmaceutical compositions of the present disclosure comprise a compound of the disclosure (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In some embodiments, the rivoceranib or a pharmaceutically acceptable salt thereof is administered parenterally. In some embodiments, the rivoceranib or a pharmaceutically acceptable salt thereof is administered intradermally, subcutaneously, intramuscularly, intraosseously, intraperitoneally, or intravenously. In some embodiments, the rivoceranib or a pharmaceutically acceptable salt thereof is administered intraperitoneally. In some embodiments, the rivoceranib or a pharmaceutically acceptable salt thereof is administered intravenously. Pharmaceutical compositions of the present disclosure suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the disclosure, and/or pharmaceutically acceptable salts thereof, can also be prepared in powder or liquid concentrate form.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

In one embodiment of the present disclosure, a composition of the rivoceranib or a pharmaceutically acceptable salt thereof of the disclosure may be administered in a single dosage form comprising all the therapeutically active agents together.

In another embodiment, the rivoceranib or a pharmaceutically acceptable salt thereof of the present disclosure comprises more than two compositions contained in the same or separate containers, and these at least two compositions may be administered separately, either simultaneously or sequentially. In another embodiment, the rivoceranib or a pharmaceutically acceptable salt thereof of the present disclosure comprises more than two compositions contained in a blister pack, and these at least two compositions may be administered separately, either simultaneously or sequentially.

### Dosing

In some instances, drug dosages are determined as a factor of patient body surface area (BSA). In some instances, BSA is a better indicator of metabolic mass than body weight because it is less affected by abnormal adipose mass, e.g., a patient with a larger BSA would presumably have larger organs for a drug to clear through. Indeed, there can be a 4-10 fold variation in drug clearance between individuals.

Various formulae exist, using height and weight, to calculate BSA without direct measurement. The most widely used is the Du Bois formula, which has been shown to be equally as effective in estimating BSA in obese and non-obese patients.

$$BSA = 0.007184 \times {W}^{0.425} \times {H}^{0.725}$$

where W is mass in kg, and H is height in cm. The average adult male BSA is 2.060 m². The average adult female BSA is 1.830 m².

In some instances, drug dosages can be determined based upon the subject's response to the drug, e.g., adverse events other than disease progression. For example, when a subject is administered a dose of the drug or multiple doses of the drug and exhibits an adverse event other than disease progression, the dosage of the drug may be reduced or the dosage of the drug may be interrupted, e.g., the dosage of the drug is no longer administered daily and can be postponed days without administration or can be administered every other day or less.

In some embodiments, the rivoceranib or a pharmaceutically acceptable salt thereof is administered once daily. In some embodiments, the rivoceranib or a pharmaceutically acceptable salt thereof is administered twice daily. In some embodiments, the rivoceranib or a pharmaceutically acceptable salt thereof is administered less than once daily in the event the subject experiences an adverse event other than disease progression.

In some embodiments, the rivoceranib or a pharmaceutically acceptable salt thereof is administered once daily in a total daily amount of at least or greater than 500 mg and up to about 1000 mg as a starting dose for the first one, two, three, four, five, six, seven, or more days. In some embodiments, the rivoceranib or a pharmaceutically acceptable salt thereof is administered once daily in a total daily amount of at least about 550 mg and up to about 700 mg. In some embodiments, the rivoceranib or a pharmaceutically acceptable salt thereof is administered once daily in a total daily amount of about 700 mg. In some embodiments, the rivoceranib or a pharmaceutically acceptable salt thereof is administered twice daily in a total daily amount of at least about 500 mg and up to about 1000 mg. In some embodiments, the total daily amount of rivoceranib or a pharmaceutically acceptable salt thereof is reduced to a total daily amount administered of about 500 mg, 400 mg, 300 mg, 200 mg, or 100 mg if the subject is experiencing serious adverse events other than disease progression or treatment emergent adverse events.

Described herein are dosing regimens for the treatment of adenoid cystic carcinoma. In some embodiments, the dosing regimen is for treating a subject for ACC, e.g., R/M ACC, wherein the subject has measurable ACC tumors/lesions that are increasing in number and/or have increased in a dimension and/or volume by 20% or more prior to treatment, or has an appearance of one or more new ACC tumors/lesions prior to treatment or administering. In some embodiments, the dosing regimen is for the treatment of R/M ACC in a subject, and the dosing regimen comprises administering to the subject a total daily amount of at least about 500 mg and up to about 1000 mg rivoceranib or a pharmaceutically acceptable salt thereof for at least one cycle, wherein each cycle is at least 10 days. In some embodiments, the rivoceranib or a pharmaceutically acceptable salt thereof is administered once daily. In some embodiments, the cycle is 28 days. In some embodiments, the cycle is 28 days or more. In some embodiments, the dosing regimen comprises administering to the subject a total daily amount of at least about 500 mg and up to about 1000 mg rivoceranib or a pharmaceutically acceptable salt thereof for at least two cycles, at least three cycles, at least four cycles, at least five cycles, or at least six cycles.

In some embodiments, the methods described herein further comprise or consist of administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, beta2-agonists, anticholinergic agents, hormonal agents, biological agents, immunotherapeutic agents, glucocorticoids, corticosteroid agents, antibacterial agents, antihistamines, anti-malarial agents, anti-viral agents, and antibiotics; and, optionally with radiation therapy.

In some embodiments, the rivoceranib or a pharmaceutically acceptable salt thereof is administered for a cycle of at least 10 days. In some embodiments, the rivoceranib or a pharmaceutically acceptable salt thereof is administered for a cycle of 28 days. In some embodiments, the rivoceranib or a pharmaceutically acceptable salt thereof is administered for at least 2 months, e.g., in multiple cycles. In some embodiments, the rivoceranib or a pharmaceutically acceptable salt thereof is administered for about 2 months. In some embodiments, the rivoceranib or a pharmaceutically acceptable salt thereof is administered for about 3 months. In some embodiments, the rivoceranib or a pharmaceutically acceptable salt thereof is administered for about 4 months. In some embodiments, the rivoceranib or a pharmaceutically acceptable salt thereof is administered for about 5 months. In some embodiments, the rivoceranib or a pharmaceutically acceptable salt thereof is administered for about 6 months. In some embodiments, the rivoceranib or a pharmaceutically acceptable salt thereof is administered for about 7 months. In some embodiments, the rivoceranib or a pharmaceutically acceptable salt thereof is administered for about 8 months. In some embodiments, the rivoceranib or a pharmaceutically acceptable salt thereof is administered for about 9 months. In some embodiments, the rivoceranib or a pharmaceutically acceptable salt thereof is administered for about 10 months. In some embodiments, the rivoceranib or a pharmaceutically acceptable salt thereof is administered for about 11 months. In some embodiments, the rivoceranib or a pharmaceutically acceptable salt thereof is administered for about 12 months. In some embodiments, the rivoceranib or a pharmaceutically acceptable salt thereof is administered for at least 2 months.

### Kits

In some embodiments, kits are provided comprising at least or greater than 500 mg and up to about 1000 mg of rivoceranib or a pharmaceutically acceptable salt thereof, wherein the kits are for treating ACC, e.g., R/M ACC, and in particular, severe R/M ACC as defined herein. In some embodiments, kits are provided comprising about 700 mg of rivoceranib or a pharmaceutically acceptable salt thereof, wherein the kits are for treating R/M ACC and in particular, severe R/M ACC.

In some embodiments, kits are provided, comprising a tablet(s) that include a total of at least or greater than 500 mg and up to about 1000 mg of rivoceranib or a pharmaceutically acceptable salt thereof, wherein the kits are for treating adenoid cystic carcinoma. In some embodiments, kits are provided, further comprising tablets that include one or more of pregelatinized starch, microcrystalline cellulose, sodium starch glycolate, povidone (K-30), colloidal silicon dioxide, magnesium stearate and Opadry white.

In some embodiments, kits are provided, comprising one or more 100 mg tablets comprising rivoceranib or a pharmaceutically acceptable salt thereof, e.g., one 100 mg tablet, two 100 mg tablets, three 100 mg tablets, four 100 mg tablets, five 100 mg tablets, six 100 mg tablets, seven 100 mg tablets, eight 100 mg tablets, nine 100 mg tablets, or ten 100 mg tablets, optionally in a blister pack (e.g., an aluminum film blister pack), wherein the kits are for treating adenoid cystic carcinoma.

In some embodiments, kits are provided, comprising one or more 200 mg tablets comprising rivoceranib or a pharmaceutically acceptable salt thereof, e.g., one 200 mg tablet, two 200 mg tablets, three 200 mg tablets, four 200 mg tablets, or five 200 mg tablet, optionally in a blister pack (e.g., an aluminum film blister pack), wherein the kits are for treating adenoid cystic carcinoma.

In some embodiments, kits are provided, comprising one or more 250 mg tablets comprising rivoceranib or a pharmaceutically acceptable salt thereof, e.g., one 250 mg tablet, two 250 mg tablets, three 250 mg tablets, or four 250 mg tablets, optionally in a blister pack (e.g., an aluminum film blister pack), wherein the kits are for treating adenoid cystic carcinoma. In some embodiments, kits are provided, comprising one or more 100 mg tablets comprising rivoceranib or a pharmaceutically acceptable salt thereof and one or more 200 mg tablets comprising rivoceranib or a pharmaceutically acceptable salt thereof, e.g., one 200 mg tablet and one 100 mg tablet; one 200 mg tablet and two 100 mg tablets; one 200 mg tablet and three 100 mg tablets; one 200 mg tablet and four 100 mg tablet; two 200 mg tablets and one 100 mg tablet; two 200 mg tablets and two 100 mg tablets; two 200 mg tablets and three 100 mg tablets; three 200 mg tablets and one 100 mg tablet; three 200 mg tablets and two 100 mg tablets; three 200 mg tablets and three 100 mg tablets; or four 200 mg tablets and one 100 mg tablet; in a blister pack (e.g., an aluminum film blister pack), wherein the kits are for treating adenoid cystic carcinoma.

In some embodiments, kits are provided, comprising three 100 mg tablets comprising rivoceranib or a pharmaceutically acceptable salt thereof and two 200 mg tablets comprising rivoceranib or a pharmaceutically acceptable salt thereof, wherein the kit is in a blister pack (e.g., an aluminum blister pack) and are for treating adenoid cystic carcinoma, e.g., wherein the subject has measurable tumors/lesions that are increasing in number, or have increased in a dimension or volume by 20% or more, or have an appearance of new lesions prior to treating.

### EXAMPLES

The disclosure includes the following examples, which do not limit the scope of the invention described in the claims.

### Example 1—Ongoing Phase II Clinical Trials of Rivoceranib

Overall design: An ongoing Phase 2, multicenter, open-label, single-arm study was designed to evaluate the efficacy and safety of rivoceranib in patients with recurrent or metastatic (R/M) adenoid cystic carcinoma (ACC) of all anatomic sites of origin. The study was composed of an initial screening phase (up to 28 days), a treatment phase, and a follow-up phase.

Objectives: Primary objectives were to evaluate safety & tolerability, efficacy by objective response rate (ORR), best overall response (BOR), time to response, duration of response, disease control rate, and duration of disease control. Secondary objectives were to evaluate efficacy as measured by overall survival, progression-free survival and event-free survival. The primary efficacy endpoint was the ORR, defined as the proportion of patients who achieved confirmed complete response (CR) or partial response (PR) per Response Evaluation Criteria in Solid Tumors (RECIST) Version 1.1 (V1.1). The secondary efficacy endpoints were duration of response (DoR) assessed by the investigator and a blinded independent review committee (BIRC), progression-free survival (PFS) assessed at 6 months, 12 months, and 2 years by investigator and BIRC, Time to Progression (TTP) assessed by investigator and BIRC, and overall survival (OS) assessed at 1 and 2 years. Exploratory efficacy endpoints include ORR per CHOI response criteria (based on the journal article, Choi et al., Correlation of computed tomography and positron emission tomography in patients with metastatic gastrointestinal stromal tumor treated at a single institution with imatinib mesylate: proposal of new computed tomography response criteria. J Clin Oncol. 2007; 25(13): 1753-1759) by a blinded independent review study (BIRC) and disease control rate (DCR) per RECIST V1.1 by Investigator and BIRC. Except for CHOI response criteria, all response endpoints were assessed per RECIST V1.1 by Investigator and BIRC.

Patients: The study was conducted at 11 study sites in the USA and South Korea. A total of 80 patients were enrolled and received at least 1 dose of study drug (rivoceranib). Fifty-three patients were enrolled in the USA and 27 patients were enrolled in Korea. Eligible patients were 18 years or older and were required to have histologically or cytologically confirmed R/M ACC of any anatomic location not amenable to curative surgery or radiotherapy. Due to the indolent nature of the disease, patients were required to have evidence of disease progression by RECIST V1.1 within the 6 months prior to study entry, evidenced by: ≥20% increase in radiologically or clinically measurable lesions or appearance of new lesions. Patients were required to have at least 1 measurable target lesion by RECIST V1.1. There were no restrictions on prior systemic therapies. Patients were permitted to have prior VEGFR TKI exposure if dosage was discontinued within 5 half-lives prior to rivoceranib treatment. The patients were permitted if they have an Eastern Cooperative Oncology Group (ECOG) performance status of 0 or 1. Patients with treated central nervous system metastases were permitted if they were stable for four weeks prior to study treatment. Eligible patients had documented primary diagnosis of solid tumor cancer of ACC for which anti-VEGFR-2 targeted therapy could be applicable. The cancer history of the patients is summarized in Table 1A below. The majority of patients had primary tumors in the major (33.8%) or minor (58.8%) salivary glands; 6 patients (7.5%) had primary tumors in other locations: lung, breast, trachea, Cowper's gland, intracranial temporal lobe and lymph node. Median time from original ACC diagnosis was 69.62 months. The majority of patients (92.5%) had metastatic disease. The baseline characteristics of the patient population are summarized in Table 1B below.

TABLE 1A
Cancer History (Safety Population, N = 80)
Original Primary Tumor Location (n, %)

| | | |
|---|---|---|
| Major salivary gland^{a} | 27 | (33.8) |
| Minor salivary gland^{b} | 47 | (58.8) |
| Other^{c} | 6 | (7.5) |

Time from Original ACC Diagnosis (months)

| N | 80 | |
|---|---|---|
| Mean (SD) | 96.38 | (87.910) |

| | |
|---|---|
| Median | 69.62 |
| Min, Max | 5.5, 486.3 |

Current Stage (n, %)

| | | |
|---|---|---|
| Stage II | 1 | (1.3) |
| Stage IVA | 2 | (2.5) |
| Stage IVB | 3 | (3.8) |
| Stage IVC | 74 | (92.5) |

Current Stage - Metastasis (n, %)

| | | |
|---|---|---|
| M0 | 5 | (6.3) |
| M1 | 74 | (92.5) |
| Missing | 1 | (1.3) |

Sites of Metastases (n, %)

| | | |
|---|---|---|
| Bone | 20 | (25.0) |
| Central Nervous System-Brain | 4 | (5.0) |
| Kidney | 6 | (7.5) |
| Liver | 25 | (31.3) |
| Lung | 69 | (86.3) |
| Lymph Nodes (Non-Regional) | 20 | (25.0) |
| Peritoneum | 7 | (8.8) |
| Pleura | 14 | (17.5) |
| Soft Tissue | 3 | (3.8) |
| Other | 9 | (11.3) |

^{a}Major salivary glands include: parotid gland and submandibular gland.
^{b}Minor salivary glands include: hard palate, soft palate, lip, tongue, tonsil, false cord, nasal cavity, paranasal sinus, tonsil, maxillary sinus, nasopharynx, ethmoid sinus, larynx, lacrimal gland, and auditory canal.
^{c}Other locations include: lung, and breast, trachea, Cowper's gland, intracranial temporal lobe and lymph node.

TABLE 1B
Baseline Characteristics of the Overall Population

| | Rivoceranib | |
|---|---|---|
| Characteristics | N = 80 | |
| Median age, y (range) | 54.5 | (28, 76) |
| Male, n (%) | 42 | (52.5) |
| ECOG PS 0, n (%) | 45 | (56.3) |
| Primary tumor location, n (%) | | |
| Major salivary gland | 27 | (33.8) |
| Minor salivary gland | 47 | (58.8) |
| Other | 6 | (7.5) |
| Stage IVC, n (%) | 74 | (92.5) |
| Sited of metastases, n (%) | | |
| Lung | 69 | (86.3) |
| Liver | 25 | (31.3) |
| Bone | 20 | (25.0) |
| Lymph nodes | 20 | (25.0) |
| Pleura | 14 | (17.5) |
| Prior surgery/radiotherapy, n (%) | 71 (88.8)/77 (96.3) | |
| Prior systemic therapy, n (%) | 49 | (61.3) |
| Median number of lines (range) | 1.0 | (0, 8) |
| ≥3 lines | 16 | (20.0) |
| Prior VEGFR inhibitor^{a} | 14 | (17.5) |
| Lenvatinib | 10 | (12.5) |
| Axitinib | 4 | (5.0) |
| Prior chemotherapy | 37 | (46.3) |

^{a}No patients received >1 line of prior VEGFR inhibitor therapy.

Prior ACC Treatment: Seventy-one patients (88.8%) had prior surgery, 77 patients (96.3%) had prior radiation therapy and 49 patients (61.3%) had prior systemic therapy. A total of 37 patients (46.3%) had prior chemotherapy (16.3% with ≥2 lines) and 14 patients (17.5%) had prior VEGF targeted therapy (all 1 line; 10 patients [12.5%] with lenvatinib and 4 patients [5.0%] with axitinib); 18 patients (22.5%) had prior treatment with monoclonal antibodies including immunotherapy.

Dose determination: Enrolled patients initiated treatment with rivoceranib 700 mg daily (QD). Up to 3 levels of dose reduction for toxicity were permitted: 500 mg QD, 300 mg QD and 200 mg QD. Patients received treatment until disease progression, unacceptable toxicity, death or withdrawal of consent to treatment. Patients deriving benefit were permitted to continue treatment with rivoceranib beyond disease progression. After discontinuation of study treatment, patients were to be followed for survival (and initiation of new anticancer therapy) for 24 months. Patients were dosed with rivoceranib 700 mg orally daily (QD) in 28-day cycles. Treatment with rivoceranib was continued until disease progression, unacceptable toxicity, death, or withdrawal of consent. Rivoceranib 700 mg was studied as a single agent in patients with adenoid cystic carcinoma and demonstrated efficacy against adenoid cystic carcinoma with manageable toxicity, establishing once daily rivoceranib 700 mg as the recommended dose for patients with adenoid cystic carcinoma.

Study Duration: Rivoceranib doses and treatment duration are summarized in Table 2 below. The median duration of treatment was 31.07 weeks (range: 0.6 to 103.0). The median time-averaged daily dose was 397.6 mg/day, which was 60.1% of initial daily dose of 700 mg/day. Overall, 85% of patients required 1 or more dose modifications (reduction or interruption) due to an adverse event (AE). A total of 61 patients (76.3%) required dose interruption due to an adverse event (AE) and 62 patients (77.5%) required dose reduction due to an AE.

TABLE 2
Rivoceranib Dose and Treatment Duration (Safety Population)

| | Rivoceranib | |
|---|---|---|
| | N = 80 | |
| Duration of Rivoceranib | | |
| Treatment (weeks) [1] | | |
| Mean (SD) | 38.51 | (31.264) |
| Median | 31.07 | |
| Min, Max | 0.6, 103.0 | |
| Number of Doses | | |
| Mean (SD) | 234.3 | (196.34) |
| Median | 202.5 | |
| Min, Max | 4, 692 | |
| Number of Cycles that Rivoceranib | | |
| Dosing was Initiated (n, %) | | |
| 1 | 8 | (10.0) |
| 2 | 9 | (11.3) |
| 3 | 5 | (6.3) |
| 4 | 4 | (5.0) |
| 5 | 2 | (2.5) |
| 6 | 6 | (7.5) |
| 7 | 3 | (3.8) |
| 8 | 5 | (6.3) |
| 9 | 1 | (1.3) |
| 10 or more | 37 | (46.3) |
| Number of Subjects with Dose | 71 | (88.8) |
| Modification (n, %) [2] [3] | | |
| Because of Adverse event | 68 | (85.0) |
| Subject error | 33 | (41.3) |
| Physician decision | 12 | (15.0) |
| Dosing error | 2 | (2.5) |
| Other | 28 | (35.0) |
| Number of Subjects with Dose | 67 | (83.8) |
| Interruption (n, %) [2] [3] | | |
| Adverse event | 61 | (76.3) |
| Subject error | 30 | (37.5) |
| Physician decision | 6 | (7.5) |
| Dosing error | 2 | (2.5) |
| Other | 27 | (33.8) |
| Number of Subjects with Dose | 66 | (82.5) |
| Reduction (n, %) [2] [3] | | |
| Adverse event | 62 | (77.5) |
| Subject error | 4 | (5.0) |
| Physician decision | 7 | (8.8) |
| Other | 2 | (2.5) |

The same subject may have more than 1 reason for dose interruption, modification, or dose reduction if multiple events occurred.
Data for interruptions and dose reductions in this table are derived from the drug administration in the eCRF and may be inconsistent with dose interruptions and dose reductions provided in TEAE tables. The latter allow only one action taken (i.e., drug interruption, dose reduction, or drug discontinuation) for each TEAE, typically the most definitive action.
[1] Exposure to rivoceranib (weeks) = (Last dose date of rivoceranib − First dose date of rivoceranib + 1)/7.
[2] The same subject may have multiple dose modifications. All dose interruptions during the treatment period (prior to last dose date) are counted.
[3] n denotes the number of subjects with dose modifications.

Study treatment details: Rivoceranib is administered as the mesylate salt of its free base, provided as 100 mg and 200 mg tablets in PVC heat-sealed, foil-laminated blister packs. Rivoceranib doses as provided in this example, are given as the amount of freebase rather than the mesylate salt. The freebase dosage is approximately 81% of the mesylate dosage. The formulation is the same. Referring to rivoceranib dose strength as freebase aligns with standards for referencing total active product.

Results: The patients enrolled in this study were a population of patients with aggressive R/M ACC; 74 (92.5%) patients had distant metastases. Their disease was not indolent based on the eligibility requirement to demonstrate progression per RECIST V1.1 criteria in the 6 months prior to study entry. The aggressiveness of their disease is further substantiated by the fact that 39 (48.5%) patients had disease progression and at least 17 (21.3%) patients died due disease progression or cancer, despite treatment within the 19.5-month follow-up of the study. The primary endpoint, ORR, was 15.1% (95% confidence interval (CI) 7.8, 25.4) by Investigator and 9.6% (95% CI 3.9, 18.8) by BIRC. Lesion response assessments in this disease are challenging due to the extent of prior surgical resection and radiotherapy to primary tumor lesions and the large number of pulmonary metastases in many patients, making selection of target lesions difficult. These challenges were substantiated by the discordance rate observed between Investigator and BIRC assessment of response. Note that ORR based on the number of patients assessed as responders by either Investigator or BIRC was 20.5% (95% CI 13.1, 32.3:15 out of 73 patients).

A total of 73 patients were evaluable for efficacy per RECIST V1.1 (defined per protocol as eligible patients who received at least 1 dose of rivoceranib and had at least 1 post baseline tumor assessment). Seven patients who did not have at least 1 post-baseline scan available were excluded from the efficacy evaluable population. Sixty patients were VEGFR inhibitor-naïve and 13 patients had prior VEGFR inhibitor therapy.

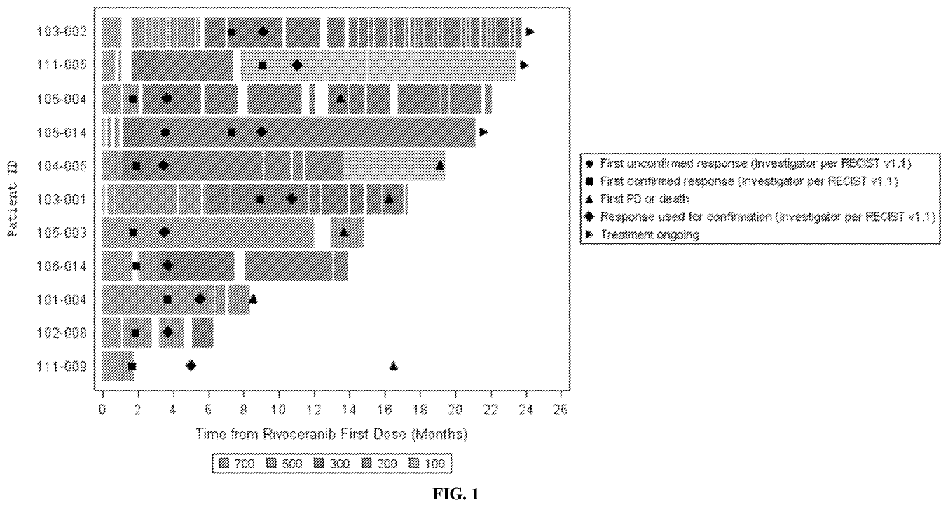

ORR, DCR, and DOR by Investigator and by BIRC using RECIST V1.1 are presented in Table 3 below. ORR was 15.1% by Investigator and 9.6% by BIRC. By Investigator, all responses occurred in patients without prior VEGFR inhibitor treatment; however, when assessed by BIRC, responses were observed in patients with (n=2, 15.4%) and without (n=5, 8.5%) prior VEGFR inhibitor therapy. Disease Control Rate, including patients with complete response (CR), partial response (PR), or stable disease (SD) of ≥3 months, was 64.4% by Investigator and 65.8% by BIRC assessments. DCRs were greater than 60% regardless of prior VEGFR inhibitor treatment. All responses were partial responses. Median DOR was 14.9 months by Investigator and 7.2 months by BIRC assessment. Median time to response by Investigator was 57.0 days (range: 50-276). Among the 11 responders, 3 remain on treatment, 5 had progressed, 2 discontinued treatment due to AE (ejection fraction decreased and tracheoesophageal fistula), and 1 withdrew consent. Of note, 1 patient (111-009) had a confirmed response approximately 3 months after discontinuing treatment due to AE (tracheoesophageal fistula) without any other treatment administered. Time to response, response confirmation and progression, and duration of response and treatment was shown for the 11 responders by Investigator assessment FIG. 1. In FIG. 1, the horizontal bar stands for the duration of treatment and was calculated as 12*(Treatment end date−Treatment start date+1)/365.25. Time to event from rivoceranib first dose was calculated as 12*(Event date−Treatment start date+1)/365.25.0 represents rivoceranib first dose date in the x-axis.

TABLE 3
Response by Investigator and by BIRC per RECIST V1.1 (Efficacy Evaluable Population)

| | Total | | VEGFRi-naïve | | VEGFRi-treated | |
|---|---|---|---|---|---|---|
| | N = 73 | | N = 60 | | N = 13 | |
| | Investigator | BIRC | Investigator | BIRC | Investigator | BIRC |
| ORR, n (%) | 11 (15.1) | 7 (9.6) | 11 (18.3) | 5 (8.3) | 0 | 2 (15.4) |
| [1] | | | | | | |
| ORR 95% CI | 7.8, 25.4 | 3.9, 18.8 | 9.5, 30.4 | 2.8, 18.4 | 0, 24.7 | 1.9, 45.4 |
| [2] | | | | | | |
| DCR, n (%) | 47 (64.4) | 48 (65.8) | 37 (61.7) | 39 (65.0) | 10 (76.9) | 9 (69.2) |
| [3] | | | | | | |
| DCR 95% CI | 52.3, 75.3 | 53.7, 76.5 | 48.2, 73.9 | 51.6, 76.9 | 46.2, 95.0 | 38.6, 90.9 |
| [2] | | | | | | |

BOR RECIST V1.1

| | | | | | | |
|---|---|---|---|---|---|---|
| Complete | 0 | 0 | 0 | 0 | 0 | 0 |
| Response, n | | | | | | |
| (%) | | | | | | |
| Partial | 11 (15.1) | 7 (9.6) | 11 (18.3) | 5 (8.3) | 0 | 2 (15.4) |
| Response, n | | | | | | |
| (%) | | | | | | |
| Stable | 54 (74.0) | 61 (83.6) | 43 (71.7) | 51 (85) | 11 (84.6) | 10 (76.9) |
| Disease, n | | | | | | |
| (%) | | | | | | |
| Progressive | 8 (11.0) | 4 (5.5) | 6 (10.0) | 3 (5.0) | 2 (15.4) | 1 (7.7) |
| Disease, n | | | | | | |
| (%) | | | | | | |
| Not | 0 | 1 (1.4) | 0 | 1 (1.7) | 0 | 0 |
| Evaluable, n | | | | | | |
| (%) | | | | | | |

DoR (months)

| | | | | | | |
|---|---|---|---|---|---|---|
| 25th | 11.9 | 5.5 | 11.9 | 5.4 | NE | 5.5 |
| percentile | | | | | | |
| Median | 14.9 (4.9, 17.3) | 7.2 (3.5, 8.4) | 14.9 (4.9, 17.3) | 9.9 (3.5, 8.4) | NE (NE, NE) | 6.3 (5.5, 7.1) |
| (95% CI) | | | | | | |
| 75th | 17.3 | 8.3 | 17.3 | 8.3 | NE | 7.1 |
| percentile | | | | | | |

BOR = Best Overall Response; DCR = Disease Control Rate; CI = Confidence Interval; ORR = Objective Response Rate; RECIST V1.1 = Response Evaluation Criteria in Solid Tumors Version 1.1; VEGFRi = vascular endothelial growth factor inhibitor.
Partial or complete response should be confirmed at the next imaging assessment to be included in the calculation of ORR and BOR.
Subjects with unconfirmed partial response (PR)/ complete response (CR) are considered to have a BOR of stable disease (SD).
[1] Subjects with a BOR of PR/CR. BOR of PR/CR must be confirmed for inclusion in the numerator of ORR.
[2] Calculated using the Clopper-Pearson method.
[3] Disease control rate is the proportion of subjects who achieve a CR, PR, or SD for ≥3 months from the start of treatment with rivoceranib and are confirmed with the same or better response at the immediate next tumor assessment.
DoR (months) = 12*(Date of Progressive Disease or death − First date of Partial Response/Complete Response + 1)/365.25.
DoR is estimated using the Kaplan-Meier method.

An additional analysis investigated the effect of any prior systemic treatment on response (including prior VEGFR inhibitors). Among the efficacy evaluable population (n=73), 44 patients (60.3%) had any prior systemic therapy for ACC and 29 patients (39.7%) had no prior systemic therapy. By Investigator assessment, ORR was 20.7% and 11.4% (Table 4) and duration of response was 14.9 months and 17.3 months for prior systemic therapy naïve and treated patients, respectively (Table 4). Disease control rate was 58.6% for patients without prior systemic treatment and 68.2% for patients with prior systemic treatment (Table 4).

TABLE 4
Response by Prior Systemic Therapy per RECIST V1.1
by Investigator (Efficacy Evaluable Population)

| | | | Systemic therapy - | | Systemic therapy - | |
|---|---|---|---|---|---|---|
| | Total | | naïve | | treated | |
| | N = 73 | | N = 29 | | N = 44 | |
| ORR, n (%) [1] | 11 | (15.1) | 6 | (20.7) | 5 | (11.4) |
| ORR 95% CI [2] | 7.8, 25.4 | | 8.0, 39.7 | | 3.8, 24.6 | |
| DCR, n (%) [3] | 47 | (64.4) | 17 | (58.6) | 30 | (68.2) |

| | | | |
|---|---|---|---|
| DCR 95% CI [2] | 52.3, 75.3 | 38.9, 76.5 | 52.4, 81.4 |

BOR by RECIST V1.1

| Complete Response, n | 0 | | 0 | | 0 | |
|---|---|---|---|---|---|---|
| % | | | | | | |
| Partial Response, n | 11 | (15.1) | 6 | (20.7) | 5 | (11.4) |
| (%) | | | | | | |
| Stable Disease, n (%) | 54 | (74.0) | 20 | (69.0) | 34 | (77.3) |
| Progressive Disease, | 8 | (11.0) | 3 | (10.3) | 5 | (11.4) |
| n (%) | | | | | | |

| | | | |
|---|---|---|---|
| Not Evaluable, n (%) | 0 | 0 | 0 |

DoR (months)

| 25th percentile | 11.9 | | 7.4 | | 14.6 | |
|---|---|---|---|---|---|---|
| Median (95% CI) | 14.9 | (4.9, 17.3) | 13.5 | (4.9, 14.9) | 17.3 | (11.9, 17.3) |

| | | | |
|---|---|---|---|
| 75th percentile | 17.3 | 14.9 | 17.3 |

Systemic therapy includes chemotherapy, immunotherapy and TKIs, including VEGFR inhibitors.
The Efficacy Evaluable Population consists of eligible subjects treated with at least one dose of rivoceranib who have at least one post baseline tumor assessment.
ORR = Objective Response Rate.
DCR = Disease Control Rate.
CI = Confidence Interval.
BOR = Best Overall Response.
RECIST V1.1 = Response Evaluation Criteria in Solid Tumors Version 1.1.
Partial or complete response should be confirmed at the next imaging assessment to be included in the calculation of ORR and BOR.
Patients with unconfirmed PR/CR are considered to have a BOR of SD.
[1] Subjects with a BOR of PR/CR. BOR of PR/CR must be confirmed for inclusion in the numerator of ORR.
[2] Calculated using the Clopper-Pearson method.
[3] Disease control rate is the proportion of subjects who achieve a CR, PR, or SD for >=3 months from the start of treatment with rivoceranib and are confirmed with the same or better response at the immediate next tumor assessment.

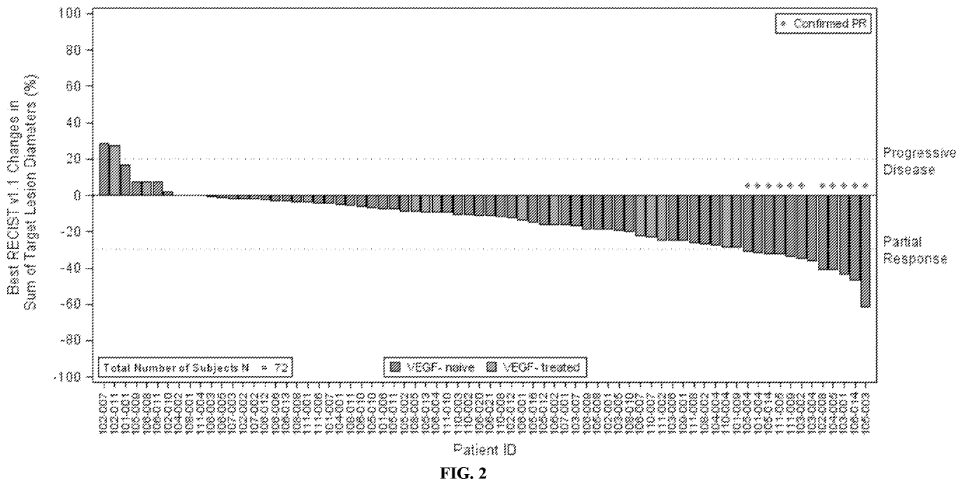

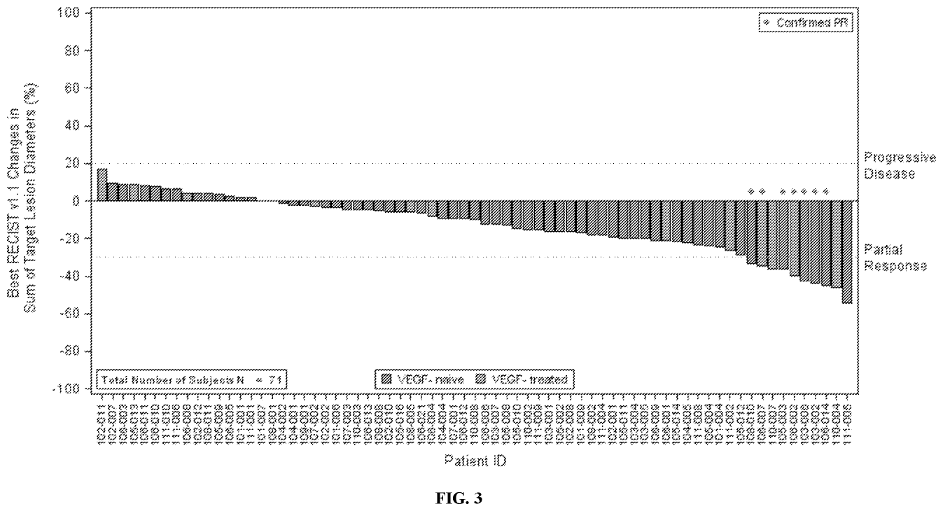

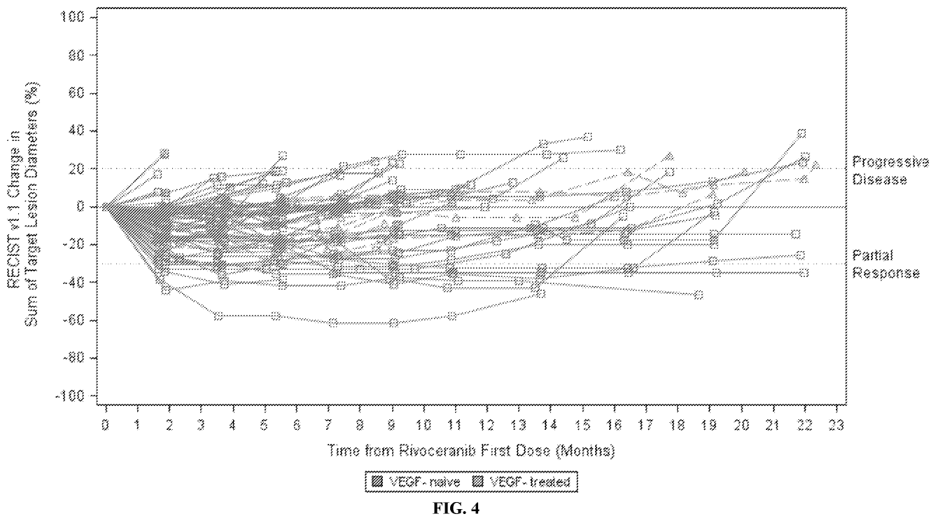

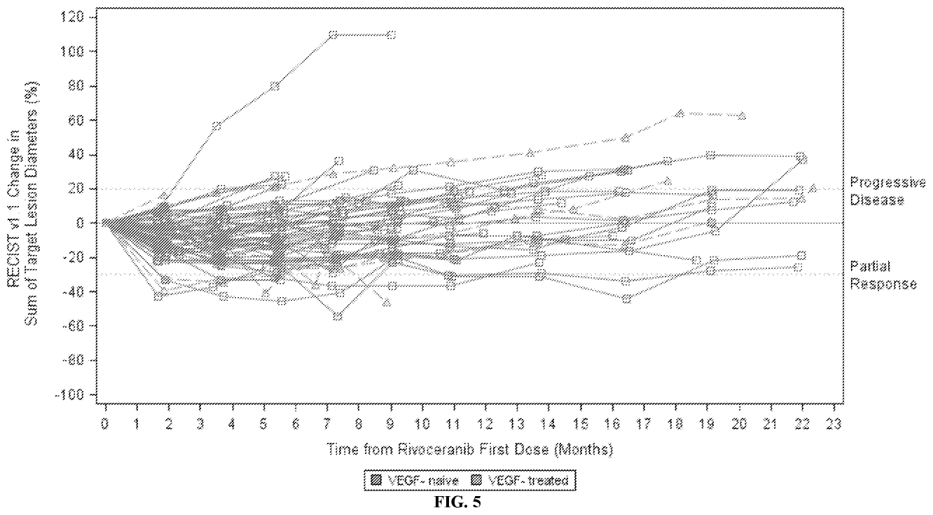

Maximum change in sum of target lesion diameters is shown by waterfall plot by Investigator and BIRC in FIG. 2 and FIG. 3, respectively, and sum of target lesions diameters over time is shown by spider plot by Investigator and by BIRC in FIG. 4 and FIG. 5, respectively. Discordance rate between Investigator and BIRC for BOR was 25% overall (n=73) and 33% for all time points (n=359). Discordance between Investigator and BIRC assessment of response may relate to the high rate of surgical resection and local radiation to primary tumors and choice of target lesions when numerous pulmonary metastases were present. All 4 figures demonstrated overall patient clinical benefit from treatment with rivoceranib as measured by reduction in tumor burden and duration over time.

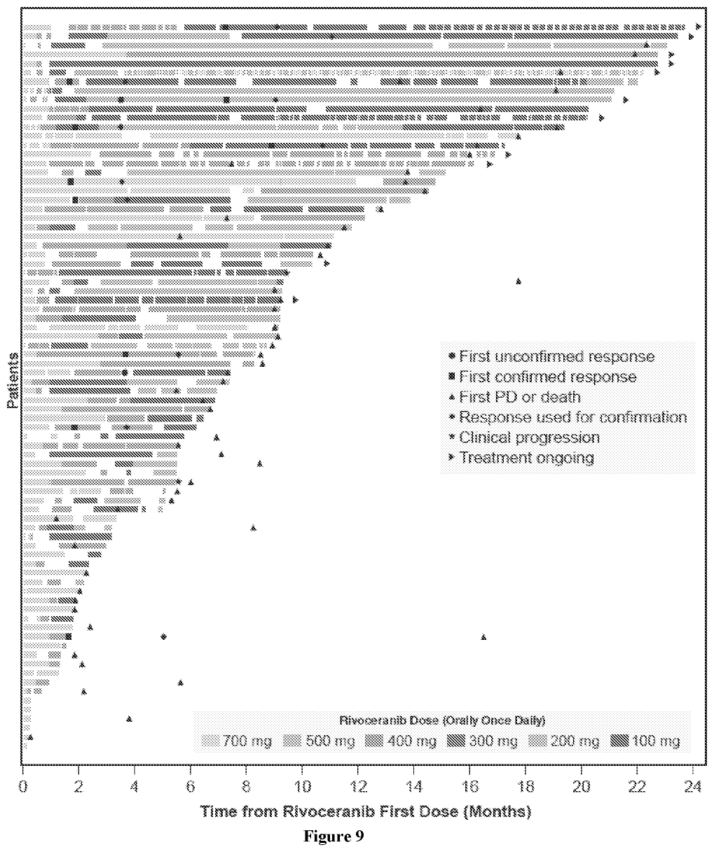

Response in Subject with Dose Reduction: 82.5% of patients had dose reductions with 80.0% of patients dose reduced from 700 mg QD to 500 mg QD; 51.3% reduced to 300 mg QD and 32.5% reduced to 200 mg QD. Median time to each of these dose reductions was 4.00 weeks, 7.71 weeks and 16.0 weeks, respectively. Efficacy appeared to be maintained despite these dose reductions. Among patients with confirmed objective response by Investigator, the initial response was observed at 700 mg in 2 patients (2.7%), at 500 mg in 4 patients (5.5%), at 300 mg in 3 patients (4.1%), at 200 mg in 1 patient (1.3%) and at 100 mg in 1 patient (%). Table 5 summarizes duration of treatment with 700 mg, 500 mg, 300 mg and 200 mg rivoceranib among the 11 patients with response by Investigator. Among the 11 patients with response, median time-averaged daily dose was 298.1 mg/day whereas the value was 397.6 mg/day for all patients. FIG. 9 summarizes the duration of treatment with 700 mg, 500 mg, 400 mg, 300 mg, 200 mg, and 100 mg rivoceranib dosages among the patients.

TABLE 5
Duration of Treatment with Each Dose in Patients
with Objective Response by Investigator

Rivoceranib
N = 11

Duration of 700 mg Treatment (months)

| | |
|---|---|
| N | 11 |
| Mean (SD) | 0.85 (0.488) |
| Median | 0.92 |
| Min, Max | 0.1, 1.7 |

Duration of 500 mg Treatment (months)

| | |
|---|---|
| N | 9 |
| Mean (SD) | 1.57 (1.622) |
| Median | 0.69 |
| Min, Max | 0.4, 4.7 |

Duration of 300 mg Treatment (months)

| | |
|---|---|
| N | 5 |
| Mean (SD) | 1.47 (0.794) |
| Median | 1.38 |
| Min, Max | 0.7, 2.8 |

Duration of 200 mg Treatment (months)

| | |
|---|---|
| N | 2 |
| Mean (SD) | 4.71 (0.488) |
| Median | 4.71 |
| Min, Max | 4.4, 5.1 |

Response on Study Compared to Pre-Study Baseline: Study eligibility required that all patients had evidence of disease progression during the 6 months prior to enrollment, documented by ≥20% increase in radiologically or clinically measurable lesions or appearance of new lesions. Unlike the general population of ACC patients who have indolent disease, patients enrolled in this study represented an enriched population of patients who had rapidly progressive disease and needed systemic therapy. Based on these eligibility criteria, 60 patients (75%) had ≥20% increase in radiologically or clinically measurable lesions and 20 patients (25%) had new lesions only; 12 patients (15%) had both ≥20% increase in radiologically or clinically measurable lesions and new lesions. Despite actively progressing disease prior to study enrollment, only 8 patients (11%) had progression, as their best response, by Investigator and 4 patients (5.5%) by BIRC.

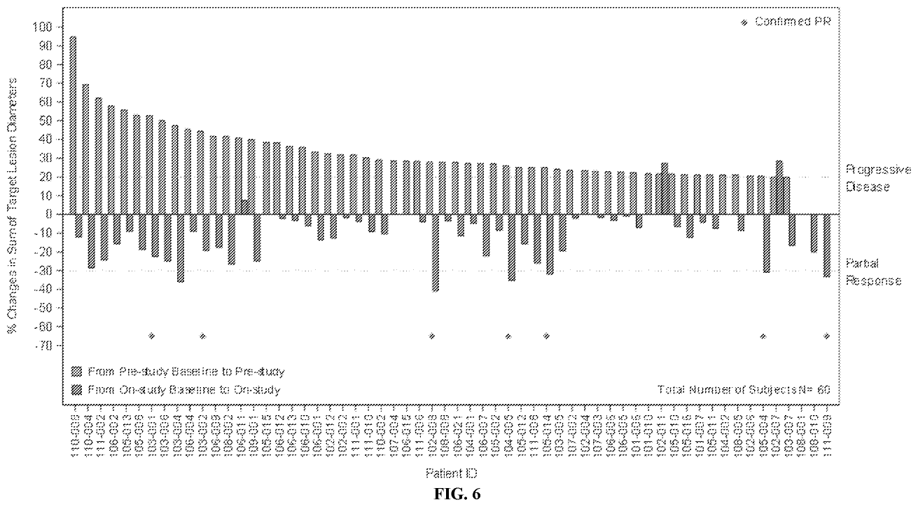

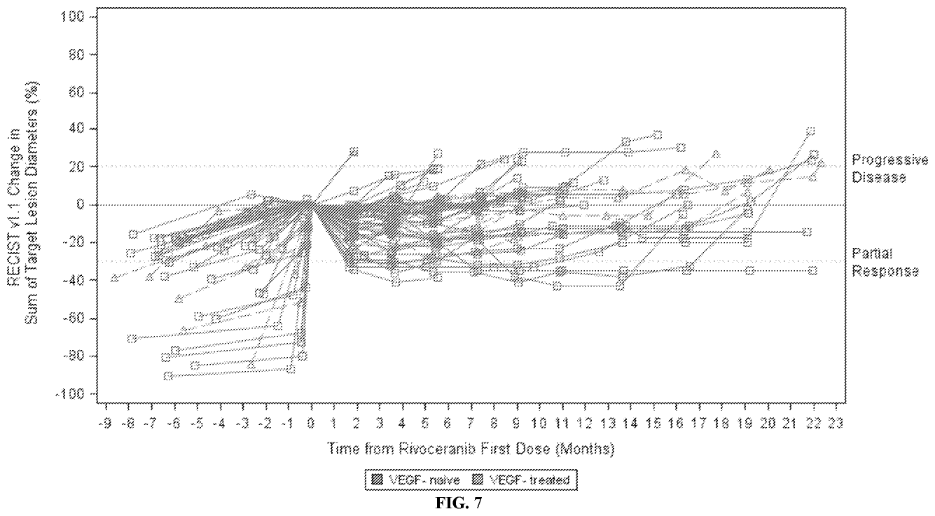

The maximum percent change in sum of target lesion diameters during the 6 months prior to enrollment and for the first 6 months of study treatment (by Investigator assessment) for the 60 patients with ≥20% increase in measurable lesions is shown in a waterfall plot (FIG. 6; n=60) and a spider plot (FIG. 7; n=51). In FIG. 6, 60 patients were noted with at least a 20% increase in radiologically or clinically measurable lesions reported by Investigator within the 6 months prior to study entry. Among these 60 patients: 3 patients (108-001, 108-010, and 111-009) did not have a pre-study baseline, and 5 patients [105-008 (13.9 months), 110-004 (10.5 months), 111-001 (22.37 months), 111-002 (33.0 months), and 111-006 (9.7 months)] had a pre-study baseline before 9 months prior to the on-study baseline, and the pre-study assessments were all within 6 months. 6 patients (101-010, 102-006, 105-015, 106-015, 107-004, and 102-004) have an on-study baseline and do not have an on-study post-baseline assessment. 51 patients had measurement of both pre-study and on-study changes in sum of target lesion diameters. In FIG. 7, 60 patients were noted with at least a 20% increase in radiologically or clinically measurable lesions reported by Investigator within the 6 months prior to study entry. There were 51 patients that had measurement of both pre-study and on-study changes in sum of target lesion diameters.

The maximum percent changes in sum of target lesion diameters (SOT) during the 6 months prior to study enrollment (Prior-SOT), during the first 6 months on treatment with rivoceranib (Post-SOT), anytime on treatment with rivoceranib (Anytime-SOT) are shown in Table 6. Median changes in Prior-SOT, Post-SOT, and Anytime-SOT were 20.5 mm (range: 5, 180), −7.0 mm (range: −43, 35, p<0.0001 compared with Prior-SOT), −8.0 mm (range: −43, 35, p<0.0001 compared with Prior-SOT). Note that 20 patients had new lesions only prior to study enrollment. Based on these data, it appeared that rivoceranib stabilized disease that was rapidly progressing prior to enrollment regardless of whether RECIST V1.1 criteria for response were met.

TABLE 6
Maximum Change in Sum of Target Lesion Diameters
for 6-Month Period Prior to Enrollment and the
6-Month Period Post Enrollment by Investigator
by RECIST V1.1 (Efficacy Evaluable Population)

| | Rivoceranib | |
|---|---|---|
| | N = 60 | |
| Maximum Change in SOT During the 6 | | |
| Months Prior to Study Enrollment (mm) | | |
| N | 52 | |
| Mean (SD) | 38.0 | (40.05) |

| | |
|---|---|
| Median | 20.5 |
| Min, Max | 5, 180 |

Maximum Change in SOT During the first 6
Months on Treatment with Rivoceranib (mm)

| N | 51 | |
|---|---|---|
| Mean (SD) | −9.9 | (13.03) |

| | |
|---|---|
| Median | −7.0 |
| Min, Max | −43, 35 |
| p-value^{a} | <0.0001 |

Maximum Change in SOT at Any Time on Treatment (mm)

| N | 51 | |
|---|---|---|
| Mean (SD) | −10.4 | (13.20) |

| | |
|---|---|
| Median | −8.0 |
| Min, Max | −43, 35 |
| p-value^{b} | <0.0001 |

SOT = Sum of target lesions diameters.
The Efficacy Evaluable Population consists of eligible subjects treated with at least 1 dose of rivoceranib who have at least 1 post baseline tumor assessment.
^{a}Paired t-test was used to compare maximum change in SOT from pre-study baseline to baseline and maximum change in SOT from baseline to best response during first 6 month on treatment. n′ = number of subjects who have both measurements for these two periods.
^{b}Paired t-test was used to compare maximum change in SOT from pre-study baseline to baseline and maximum change in SOT from baseline to best response anytime on treatment. n′ = number of subjects who have both measurements for these two periods.

Evaluation of PFS, TTP, and OS: Median PFS by Investigator assessment estimated by the Kaplan-Meier method was 9.0 months. The estimated PFS rates at 6, 9, and 12 months were 76.3%, 53.6% and 39.7%, respectively. Median PFS by BIRC assessment estimated by the Kaplan-Meier method was 9.2 months. The estimated PFS rates at 6, 9, and 12 months were 80.8%, 53.5% and 38.2%, respectively. PFS was calculated by PFS (months)=12*(Date of Progressive Disease or death−First dose of rivoceranib+1)/365.25. In general, PFS was similar regardless of prior VEGFR inhibitor treatment. In the subgroup analysis for prior systemic anticancer therapy, median PFS estimated by the Kaplan-Meier method was 13.7 months in patients who had not received any prior systemic therapy and 8.9 months in patients who had been previously treated with systemic therapy. Systemic therapy includes chemotherapy, immunotherapy and TKIs, including VEGFR inhibitors.

Median TTP by Investigator assessment was 9.1 months. The estimated TTP rates at 6, 9, and 12 months were 76.2%, 57.0% and 43.7%, respectively. TTP was calculated by TTP (months)=12*(Date of Progressive Disease−First dose of rivoceranib+1)/365.25. Median TTP by BIRC assessment was 9.3 months. The estimated TTP rates at 6, 9, and 12 months were 80.7%, 57.3% and 41%, respectively. In general, TTP was similar regardless of prior VEGFR inhibitor treatment or prior systemic therapy treatment.

Overall survival for the intention to treat (ITT) population: OS rates at 6, 9, and 12 months were 94.7%, 83.5% and 74.5%, respectively. The OS was calculated by Overall Survival (months)=12*(Date of death−First dose of rivoceranib+1)/365.25. Median OS were not evaluable. In general, OS rates at 6, 9 and 12 months were similar regardless of prior VEGFR inhibitor treatment or prior systemic anticancer treatment.

Endpoint of Response per CHOI response Criteria: CHOI response criteria assesses response based on change in tumor density as well as change in tumor size and have been most often used to assess response to treatment with tyrosine kinase inhibitors such as imatinib in gastrointestinal stromal tumors (GIST) where RECIST criteria underestimate response. Evaluation of response per CHOI response criteria by BIRC was added to the protocol for exploratory analysis because Investigators observed decreases in tumor density on treatment with rivoceranib without changes in tumor lesion diameters. Changes in tumor density had not been reported with other treatments investigated for R/M ACC.

Objective response rate (ORR), duration of response (DOR) and disease control rate (DCR) in the efficacy evaluable population per CHOI response criteria (using the same data set as used for analyses per RECIST V1.1): Assessment of response per CHOI response criteria required a subject have contrast-enhanced CT scan as the imaging modality for assessment of tumor density at baseline and post-baseline time points. Therefore, fewer subjects were efficacy evaluable per CHOI response criteria (N=60) than per RECIST V1.1 criteria (N=73). Objective response rate per CHOI response criteria was 51.7% overall, and similar ORRs were observed for the VEGFR inhibitor-naïve and VEGFR inhibitor-treated subjects (52.1% and 50.0%, respectively)—all partial responses (PR). This is >4-fold higher than the ORR of 9.6% per RECIST V1.1 reported by BIRC. Disease control rate per CHOI response criteria was 61.7%, similar to the DCR per RECIST V1.1 reported by BIRC-65.8%. Median DOR per CHOI response criteria was 14.8 months, which was two-fold higher than that reported per RECIST V1.1-7.2 months reported by BIRC.

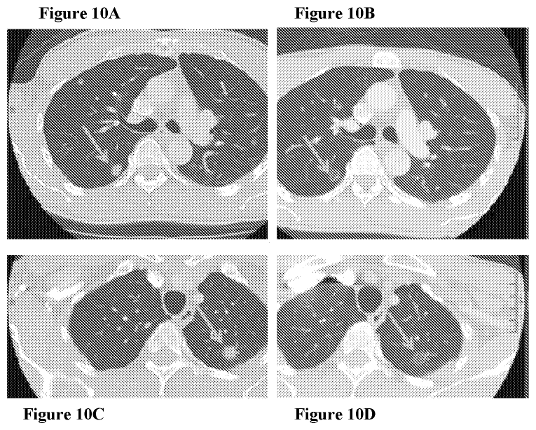

Table 7 below summarizes the response per CHOI response criteria by BIRC. FIGS. 10A-10D show an example of CT scan images of the reduction in tumor lesion density without reduction in tumor lesion size two months after starting treatment with rivoceranib in a patient.

TABLE 7
Response per CHOI Response Criteria by BIRC

| | Total | | VEGFRi-naïve | | VEGFRi-treated | |
|---|---|---|---|---|---|---|
| | N = 60 | | N = 48 | | N = 12 | |
| ORR, n (%) ^{[1]} | 31 | (51.7) | 25 | (52.1) | 6 | (50.0) |
| ORR 95% CI ^{[2]} | 38.4, 64.8 | | 37.2, 66.7 | | 21.1, 78.9 | |
| DCR, n (%) ^{[3]} | 37 | (61.7) | 29 | (60.4) | 8 | (66.7) |
| DCR 95% CI ^{[2]} | 48.2, 73.9 | | 45.3, 74.2 | | 34.9, 90.1 | |
| Complete Response, | 0 | | 0 | | 0 | |
| n (%) | | | | | | |
| Partial Response, n | 31 | (51.7) | 25 | (52.1) | 6 | (50.0) |
| (%) | | | | | | |
| Stable Disease, n | 25 | (41.7) | 20 | (41.7) | 5 | (41.7) |
| (%) | | | | | | |
| Progressive Disease, | 4 | (6.7) | 3 | (6.3) | 1 | (8.3) |
| n (%) | | | | | | |

DoR (months)

| 25th percentile | 7.2 | | 7.2 | | 12.2 | |
|---|---|---|---|---|---|---|
| Median (95% CI) | 14.8 | (7.4, 20.2) | 14.8 | (7.2, 20.2) | 12.2 | (5.2, NE) |

| | | | |
|---|---|---|---|
| 75^{th} percentile | 20.2 | 20.2 | NE |

ORR = Objective Response Rate.
DCR = Disease Control Rate.
CI = Confidence Interval.
BOR = Best Overall Response.
VEGFRi = vascular endothelial growth factor receptor inhibitor.
The CHOI Response Criteria Population consists of all eligible subjects treated with at least one dose of rivoceranib who have at least one baseline CT assessment and at least one evaluable post-baseline tumor assessment with contrast CT.
Partial or complete response should be confirmed at the next imaging assessment to be included in the calculation of ORR and BOR.
Patients with unconfirmed PR/CR are considered to have a BOR of SD.
^{[1]} Subjects with a BOR of PR/CR. BOR of PR/CR must be confirmed for inclusion in the numerator of ORR.
^{[2]} Calculated using the Clopper-Pearson method.
^{[3]} Disease control rate is the proportion of subjects who achieve a CR, PR, or SD for >=3 months from the start of treatment with rivoceranib and are confirmed with the same or better response at the immediate next tumor assessment.
Median PFS was 13.8 months. This is 50% longer than the median PFS of 9.2 months per RECIST V1.1 reported by BICR. The estimated PFS rates per CHOI response criteria at 6, 9, and 12 months were 80.4%, 62.6% and 53.8%, respectively. In general, PFS was similar regardless of prior VEGFR inhibitor treatment.
Median TTP per CHOI response criteria was 14.4 months. This is >50% higher than the median TTP of 9.3 months per RECIST V1.1 reported by BICR. The estimated TTP rates at 6, 9, and 12 months were 82%, 67.7% and 58.3%, respectively. In general, TTP was similar regardless of prior VEGFR inhibitor treatment.

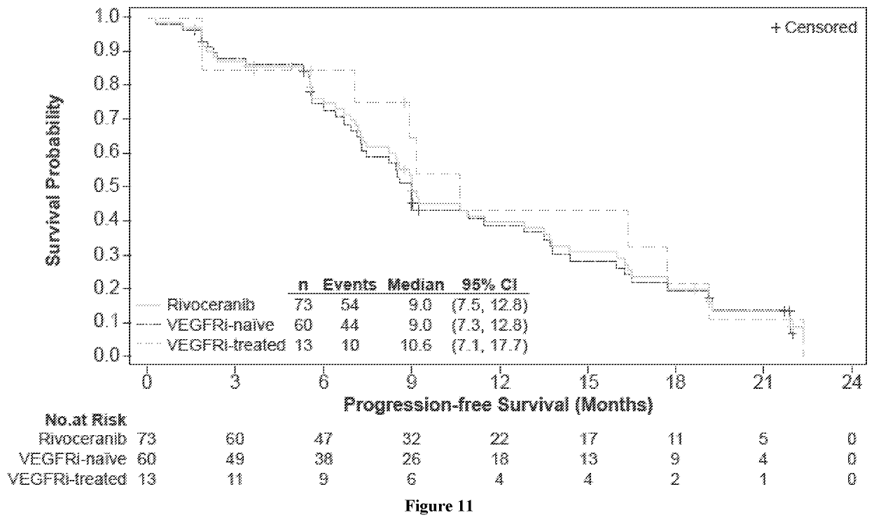

Progression-free survival (PFS) and time to progression (TTP) in the efficacy evaluable population per CHOI response criteria: Median PFS was 13.8 months. This was 50% longer than the median PFS of 9.2 months per RECIST V1.1 reported by BICR. The estimated PFS rates per CHOI response criteria at 6, 9, and 12 months were 80.4%, 62.6% and 53.8%, respectively. In general, PFS was similar regardless of prior VEGFR inhibitor treatment. The PFS per RECIST v1.1 assessed by investigator and BIRC in the efficacy evaluable population and according to prior VEGFR inhibitor treatment is summarized in Table 8 below. The Kaplan-Meier method estimates of PFS per RECIST v1.1 assessed by the investigator in the efficacy evaluable population is shown in FIG. 11.

TABLE 8
Progression-free Survival per RECIST v1.1 Assessed by Investigator and Blinded Independent Review
Committee in the Efficacy Evaluable Population and According to Prior VEGFR Inhibitor Treatment

| | Total | | VEGFRi-naïve | | VEGFRi-treated | |
|---|---|---|---|---|---|---|
| | N = 73 | | N = 60 | | N = 13 | |
| | Investigator | BIRC | Investigator | BIRC | Investigator | BIRC |
| Median, | 9.0 (7.5-12.8) | 9.2 (8.0, 12.9) | 9.0 (7.3-12.8) | 9.3 (8.0-13.7) | 10.6 (7.1-17.7) | 8.9 (5.3-12.9) |
| PFS, months | | | | | | |
| (95% CI) | | | | | | |

PFS landmark analyses, % (95% CI)

| | | | | | | |
|---|---|---|---|---|---|---|
| 6-months | 76.3 (64.2-84.8) | 80.8 (68.5-88.6) | 74.5 (60.7-84.1) | 80.3 (66.3-88.9) | 84.6 (51.2-95.9) | 83.1 (47.2-95.5) |
| 12-months | 39.7 (27.4-51.6) | 38.2 (25.0-51.3) | 38.7 (25.5-51.8) | 40.4 (25.6-54.6) | 43.0 (13.8-69.8) | 26.0 (4.3-56.2) |
| Median | 9.1 (8.5-13.8) | 9.3 (8.5-13.6) | 9.0 (7.3-13.5) | 10.8 (8.5-13.8) | 16.4 (8.9-19.1) | 8.9 (5.3-12.9) |
| TTP, months | | | | | | |
| (95% CI) | | | | | | |

Median TTP per CHOI response criteria was 14.4 months. This was >50% higher than the median TTP of 9.3 months per RECIST V1.1 reported by BIRC. The estimated TTP rates at 6, 9, and 12 months were 82%, 67.7% and 58.3%, respectively. In general, TTP was similar regardless of prior VEGFR inhibitor treatment. PFS was calculated by PFS (months)=12*(Date of progressive disease or death−First dose of rivoceranib+1)/365.25. TTP was calculated by TTP (months)=12*(Date of progressive disease−First dose of rivoceranib+1)/365.25.

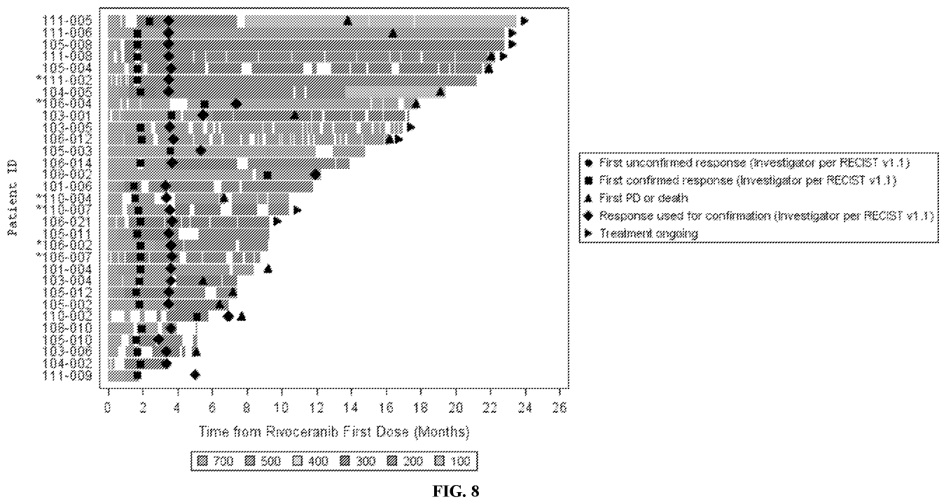

A swimmer plot for the 31 subjects with confirmed response per CHOI response criteria is shown in FIG. 8. Most responses were observed at the first tumor assessment at 8 weeks and confirmed at the second tumor assessment at 16 weeks. The horizontal bar stands for the duration of treatment. It was calculated as 12*(Treatment end date−Treatment start date+1)/365.25. Time to event from Rivoceranib first dose was calculated as 12*(Event date−Treatment start date+1)/365.25.0 represents Rivoceranib first dose date in the x-axis.

Overall survival in the intention-to-treat population according to prior VEGFR inhibitor treatment is shown in Table 9 below.

TABLE 9
Overall Survival in the Intention-to-treat Population and According to Prior
VEGFR Inhibitor Treatment

| | Total | VEGFRi-naïve | VEGFRi-treated | |
|---|---|---|---|---|
| | N = 80 | N = 66 | N = 14 | |
| Median OS, months | NE | NE | 17.5 | (10.0, NE) |
| (95% CI) | | | | |

OS landmark analyses, % (95% CI)

| | | | | |
|---|---|---|---|---|
| 12-months | 74.5 (62.5-83.1) | 77.4 (64.2-86.2) | 62.9 | (32.3-82.6) |
| 18-months | 61.7 (48.3-72.5) | 66.0 (51.1-77.3) | 45.8 | (18.3-69.9) |

In summary, patient benefit was supported by waterfall and spider plots of sum of target lesion diameters (FIG. 2-FIG. 7), both by Investigator and by BIRC, as well as the DCR of >60% by both Investigator and by BIRC, regardless of prior treatment, median duration of response of 14.9 months by Investigator and 7.2 months by BIRC, and median PFS of 9.0 and 9.2 months by Investigator and by BIRC, respectively. These are all especially noteworthy since all subjects had progressed in the prior 6 months and there were statistically significant differences (p<0.0001) in change in sum of target lesion diameters during the 6 months prior to baseline and the first 6 months on treatment. These data substantiated that progression was reversed or at least slowed by treatment with rivoceranib. All efficacy endpoints per CHOI response criteria also support patient benefit. All measures of efficacy per CHOI response criteria, except DCR which remained stable, were greater compared to RECIST criteria: ORR (9.6% vs. 50.8%), DOR (7.2 months vs. 14.8 months), DCR (65.8% vs. 60.7%), PFS (9.2 months vs. 13.8 months) and TTP (9.3 months vs. 14.4 months). It was also noteworthy that despite dose reductions, responses were observed in subjects with short durations of treatment at the initial dose level of 700 mg and mean time-averaged daily dose of 397.6 mg/day (298.1 mg/day in the 11 responding patients), suggesting that lower doses of rivoceranib may be effective in maintaining response.

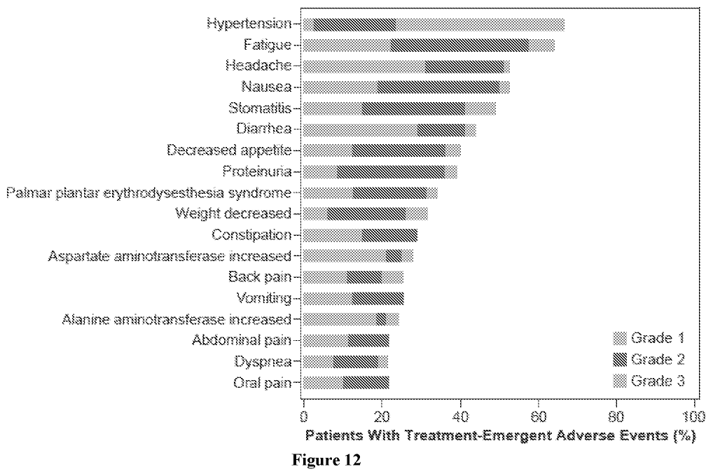

Safety Summary: Rivoceranib was generally well-tolerated with the toxicity profile as expected based on other studies of rivoceranib in patients with solid tumors, and consistent with the class effect of angiogenesis inhibitors. An adverse event of any grade was observed in all patients. Treatment-emergent adverse events in ≥20% of patients are shown in FIG. 12. Grade ≥3 adverse events were observed in 64 patients (80%). The most frequently reported treatment-emergent adverse events (TEAEs) (≥25%) were hypertension (66.3%), fatigue (63.8%), nausea and headache (52.5% each), stomatitis (48.8%), diarrhea (43.8%), decreased appetite (40.0%), proteinuria (38.8%), palmar plantar erythrodysesthesia syndrome (33.8%), weight decreased (31.3%), constipation (28.8%), aspartate aminotransferase increased (27.5%) and back pain and vomiting (25.0% each). The Grade ≥3 TEAEs reported in ≥5% of subjects were hypertension (42.5%), stomatitis (7.5%), anemia and fatigue (6.3% each), and back pain, pneumothorax, and weight decreased (5% each). Four patients experienced a fatal adverse event. These events consisted of epistaxis in two patients and acute respiratory failure in two patients. Treatment reductions and interruptions due to adverse events (AEs) were reported in 48.8% and 86.3% of patients, respectively, and 20.0% of patients discontinued treatment due to an AE. Overall, 80.0% of patients were dose reduced from 700 mg QD to 500 mg QD; 51.3% were reduced to 300 mg QD and 32.5% were reduced to 200 mg QD. The median time to each of these dose reductions was 4.00, 7.71 and 16.00 weeks, respectively. The median duration of treatment was 31.1 weeks (range, 0.6-103.0). The median actual dose intensity was 421.0 mg/day. The relative dose intensity was 60.1%. Overall, 68 patients required one or more dose modifications (reduction or interruption) due to an adverse event. The median time to first dose reduction was 4.1 weeks (range, 1-40).

Conclusions: This study was designed to evaluate efficacy and safety of rivoceranib in patients with R/M ACC who have no approved therapeutic options. A single-arm, open-label Phase 2 trial, was conducted using the Phase 2 Dose of rivoceranib of 700 mg QD. The design was robust with 11 study sites in US and Korea, with 80 patients enrolled, and the primary endpoint of ORR assessed by both Investigator and by BIRC—the first and only study, to our knowledge, to use a BIRC to assess response in patients with ACC. ORR using CHOI response criteria was also assessed. The current study took into consideration prior trials and the indolent nature of the disease and enrolled only R/M ACC patients that had progressed by RECIST criteria within 6 months prior to study entry. This represented a population with actively progressing disease in need of treatment and served as a comparator for response on treatment. The eligibility criteria were broad, allowing patients with and without prior systemic therapy, including prior VEGFR inhibitor therapy. The patients enrolled in this study had an aggressive R/M ACC disease. The primary endpoint, ORR, was 15.1% (95% CI 7.8, 25.4) by Investigator and 9.6% (95% CI 3.9, 18.8) by BIRC, respectively, and the remaining about 85% of patients had a tumor size reduction. ORR based on the number of patients assessed as responders by either Investigator or BIRC was 20.5% (95% CI 13.1, 32.3:15 out of 73 patients). The ORR observed was clinically relevant. About 52% of patients reported a response according to the CHOI response criteria which is believed to be more correlated with median overall survival that RECIST v1.1. Importantly, in a patient population that was rapidly progressing by objective criteria prior to study entry, rivoceranib reversed or slowed progression, as documented by a DCR and PFS, and DOR for patients with response. The clinically meaningful benefit was further substantiated by the changes in tumor lesion diameters by Investigator and by BIRC together with exploratory evaluation per CHOI response criteria. In conjunction with a tolerable safety profile, these data provided a favorable benefit/risk profile to support the registration of rivoceranib in patients with R/M ACC regardless of prior therapy.

This study was the largest multicenter trial of VEGFR TKI therapy in patients with R/M ACC, the first trial to require progression within 6 months prior to the trial, solely per RECIST v1.1, and the first trial to report ORR using BIRC. In patients with R/M ACC, more than half of whom had received prior systematic therapy, rivoceranib demonstrated the following: investigator-assessed ORR of 15.1%, median DOR 14.9 months, median PFS of 9 months, and disease control for >3 months in over 60% of patients, regardless of prior VEGFRi therapy; BICR-assessed ORR of 9.6%, median DOR 7.2 months, and a DCR of 64.4%; and CHOI response criteria-assessed ORR of 51.7%, median DOR 14.8 months, and a DCR of 61.7%. Regardless of previous VEGFR therapy, a PFS of 9 months was observed versus previous published data of a baseline of 2.8 months for R/M ACC. The change in sum of target lesions for the 6-month period prior to enrollment and the first 6 months on study demonstrated the efficacy of rivoceranib in this progressing population. The safety profile of rivoceranib was manageable, with an AE profile consistent with other VEGFR TKIs.

### Comparative Example 1

A single-institution trial was conducted in China with apatinib at a dose of 500 mg QD in 68 patients with R/M ACC (Zhu et al., Apatinib in patients with recurrent or metastatic adenoid cystic carcinoma of the head and neck: a single-arm, phase II prospective study. Ther Adv Med Oncol., 2021 May 8; 13:17588359211013626).

Unlike other trials, the population was limited to primary tumors involving the head and neck and only 66.2% of enrolled patients were reported to have metastatic disease, suggesting that this population of patients would fare better. Tumor response was assessed by RECIST V1.1. Primary endpoint was 6-month PFS, which was reported as 92.3%; ORR was 46.2% with median DOR of 17.7 months.

Differences in ethnicity, extent of disease at entry, study design and other unknown factors may account for the response rate and PFS that exceed results reported in other clinical trials with a TKI.

### OTHER EMBODIMENTS

While various embodiments of the present disclosure have been shown and described herein, such embodiments are provided by way of example only. Numerous variations, changes, and substitutions will now occur to those skilled in the art without departing from the inventions recited in the following claims. It should be understood that various alternatives to the embodiments of the disclosure can be employed in practice. It is intended that the following claims define the scope of the invention and that methods and structures within the scope of the claims and their equivalents be covered thereby.

What is claimed is:

1. A method of treating a subject having recurrent or metastatic adenoid cystic carcinoma (R/M ACC), the method comprising:
assessing a level of severity of the R/M ACC of the subject; and
administering to the subject a starting dosage of a total daily amount of greater than 500 mg to about 1000 mg rivoceranib or a pharmaceutically acceptable salt thereof, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over time prior to treatment as described herein, e.g., over 1, 2, 3, 4, 5, or 6 months prior to treatment.

2. The method of claim 1, wherein the subject is administered the starting dosage of the total daily amount of greater than 500 mg to about 700 mg rivoceranib or a pharmaceutically acceptable salt thereof.

3. The method of claim 1 or 2, wherein the subject is administered the starting dosage of the total daily amount of about 700 mg rivoceranib or a pharmaceutically acceptable salt thereof.

4. The method of any one of claims 1-3, wherein the rivoceranib or a pharmaceutically acceptable salt thereof is a rivoceranib mesylate salt, hydrochloride salt, sulfate salt, phosphate salt, maleate salt, citrate salt, or succinate salt.

5. The method of any one of claims 1-4, wherein the rivoceranib or a pharmaceutically acceptable salt thereof is a rivoceranib mesylate salt.

6. The method of any one of claims 1-5, wherein the rivoceranib or a pharmaceutically acceptable salt thereof is administered orally.

7. The method of any one of claims 1-6, wherein the rivoceranib or a pharmaceutically acceptable salt thereof is administered in tablet form.

8. The method of any one of claims 1-7, wherein the rivoceranib or a pharmaceutically acceptable salt thereof is administered as three 200 mg rivoceranib tablets and one 100 mg rivoceranib tablet.

9. The method of any one of claims 1-8, wherein the rivoceranib or a pharmaceutically acceptable salt thereof is administered once daily.

10. The method of any one of claims 1-9, wherein the R/M ACC is recurrent ACC.

11. The method of any one of claims 1-9, wherein the R/M ACC is metastatic ACC.

12. The method of any one of claims 1-11, wherein the rivoceranib or a pharmaceutically acceptable salt thereof is administered daily for 28 days or more.

13. The method of any one of claims 1-12, wherein the total daily amount of rivoceranib or a pharmaceutically acceptable salt thereof is down-titrated to a total daily amount of about 500 mg, about 300 mg, or about 200 mg of rivoceranib or a pharmaceutically acceptable salt thereof, if the subject is experiencing serious adverse events.

14. The method of any one of claims 1-13, further comprising discontinuing administration of rivoceranib or a pharmaceutically acceptable salt thereof if the subject no longer has any measurable ACC tumors/lesions that are increasing in number and/or have increased in a dimension and or volume by 20% or more over a 1, 2, 3, 4, 5, or 6 month period, and has had no appearance of any new tumors/lesions over a 1, 2, 3, 4, 5, or 6 month period.

15. The method of any one of claims 1-14, wherein the subject had received VEGFR inhibitor therapy prior to treatment.

16. A dosing regimen of rivoceranib or a pharmaceutically acceptable salt thereof for treatment of recurrent or metastatic adenoid cystic carcinoma (R/M ACC) in a subject, comprising administering to the subject a starting dosage of a total daily amount of greater than 500 mg and up to about 1000 mg of rivoceranib or a pharmaceutically acceptable salt thereof for at least one cycle of at least 10 days, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over time prior to treatment as described herein, e.g., over 1, 2, 3, 4, 5, or 6 months prior to treatment.

17. The dosing regimen of claim 16, wherein the rivoceranib or a pharmaceutically acceptable salt thereof is administered once daily.

18. The dosing regimen of claim 16 or 17, wherein each cycle is 28 days.

19. The method of any one of claim 16-18, wherein the subject is administered the starting dosage of a total daily amount of greater than 500 mg to about 700 mg rivoceranib or a pharmaceutically acceptable salt thereof.

20. The method of any one of claims 16-19, wherein the subject is administered the starting dosage of a total daily amount of about 700 mg rivoceranib or a pharmaceutically acceptable salt thereof.

21. A method of treating recurrent or metastatic adenoid cystic carcinoma (R/M ACC) in a subject, the method comprising:
orally administering a starting dosage of a total daily amount of about 700 mg of rivoceranib or a mesylate salt thereof to the subject,
wherein the starting dosage comprises one or more 100 mg tablets and one or more 200 mg tablets for the total daily amount of about 700 mg of rivoceranib or a mesylate salt thereof;
wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over time prior to treatment as described herein, e.g., over 1, 2, 3, 4, 5, or 6 months prior to treatment; and
wherein the total daily amount of rivoceranib or a pharmaceutically acceptable salt thereof is down-titrated to a total daily amount of about 500 mg, about 300 mg, or about 200 mg of rivoceranib or a mesylate salt thereof, if the subject is experiencing serious adverse events.

22. The method of claim 21, wherein each tablet has a core and a coating, wherein the core comprises one or more of colloidal silicone dioxide, microcrystalline cellulose, magnesium stearate, povidone, pregelatinized starch 1500, and sodium starch glycolate.

23. The method of claim 22, wherein the coating comprises one or more of hypromellose, polyethylene glycol, polysorbate 80, and titanium dioxide.

24. Use of a composition comprising a starting dosage of a total daily amount of greater than 500 mg to about 1000 mg of rivoceranib or a pharmaceutically acceptable salt thereof, for treating recurrent or metastatic adenoid cystic carcinoma (R/M ACC) in a subject, wherein the subject has one or more measurable ACC tumors/lesions that have increased in number and/or have increased in a dimension and/or volume by 20% or more prior to treatment, and/or has an appearance of one or more new ACC tumors/lesions prior to treatment, e.g., as measured or monitored over time prior to treatment as described herein, e.g., over 1, 2, 3, 4, 5, or 6 months prior to treatment.

25. The use of claim 24, wherein the starting dosage is the total daily amount of greater than 500 mg to about 700 mg of rivoceranib or a pharmaceutically acceptable salt thereof.

26. The use of claim 24 or claim 25, wherein the starting dosage is the total daily amount of about 700 mg rivoceranib or a pharmaceutically acceptable salt thereof.

27. The use of any one of claims 24-26, wherein rivoceranib or a pharmaceutically acceptable salt thereof is a rivoceranib mesylate salt, hydrochloride salt, sulfate salt, phosphate salt, maleate salt, citrate salt, or succinate salt.

28. The use of any one of claims 24-27, wherein the rivoceranib or a pharmaceutically acceptable salt thereof is a rivoceranib mesylate salt.

29. The use of any one of claims 24-28, wherein the rivoceranib or a pharmaceutically acceptable salt thereof is formulated for oral administration.

30. The use of any one of claims 24-29, wherein the rivoceranib or a pharmaceutically acceptable salt thereof is in tablet form.

31. The use of any one of claims 24-30, wherein the R/M ACC is recurrent ACC.

32. The use of any one of claims 24-30, wherein the R/M ACC is metastatic ACC.

33. The use of any one of claims 24-32, wherein the total daily amount of rivoceranib or a pharmaceutically acceptable salt thereof is down-titrated to a total daily amount of about 500 mg, about 300 mg, or about 200 mg of rivoceranib or a pharmaceutically acceptable salt thereof, if the subject is experiencing serious adverse events.
